SEQUENCE LISTING (76 sequences)

SEQ ID NO: 1 - DNA, 1347 bp, synthetic construct
  misc_feature 1..1347: note = antibody
  source 1..1347: mol_type = other DNA
  CDS 1..1347: protein_id = 65; transl_table = 1; translation = EVQLVESGGGLVQPGGSLRLSCAASGFVFSSDDMSWVRQAPGKG LEWVAYISSGGDYTYYPDTVKGRFTISRDNAKNSLYLQMNSLRAEDTAVYYCARHLYG SSSYYVMDYWGQGTLVTVSSASTKGPSVFPLAPCSRSTSESTAALGCLVKDYFPEPVT VSWNSGALTSGVHTFPAVLQSSGLYSLSSVVTVPSSNFGTQTYTCNVDHKPSNTKVDK TVERKCCVECPPCPAPPVAGPSVFLFPPKPKDTLMISRTPEVTCVVVDVSHEDPEVQF NWYVDGVEVHNAKTKPREEQFNSTFRVVSVLTVVHQDWLNGKEYKCKVSNKGLPAPIE KTISKTKGQPREPQVYTLPPSREEMTKNQVSLTCLVKGFYPSDIAVEWESNGQPENNY KTTPPMLDSDGSFFLYSKLTVDKSRWQQGNVFSCSVMHEALHNHYTQKSLSLSPGK
gaggtgcagttggttgaatctggcggaggactggttcagcctggcggatctctgagactgtcttgtgccgcctctggcttcgtgttcagctccgacgatatgtcctgggtccgacaggctcctggcaaaggattggagtgggtcgcctacatctcttctggcggcgactacacctactatcccgacaccgtgaagggcagattcaccatctccagagacaacgccaagaactccctgtacctgcagatgaacagcctgagagccgaggacaccgccgtgtactactgtgccagacacctgtacggctcctccagctactacgtgatggactattggggccagggcaccctggtcacagtgtcctctgcttccaccaagggacccagcgttttccctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctctggcctgtactctctgtcctccgtcgtgaccgtgccttcctctaactttggcacccagacctacacctgtaatgtggaccacaagccttccaacaccaaggtggacaagaccgtggaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtctcacgaggatcccgaagtgcagttcaattggtacgtggacggcgtggaagtgcacaacgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgcctcgtgaagggcttctacccttccgatatcgccgtggaatgggagagcaatggccagcctgagaacaactacaagaccacacctcctatgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaatga

SEQ ID NO: 2 - protein, 448 aa, synthetic construct
  REGION 1..448: note = Synthetic Construct
EVQLVESGGGLVQPGGSLRLSCAASGFVFSSDDMSWVRQAPGKGLEWVAYISSGGDYTYYPDTVKGRFTISRDNAKNSLYLQMNSLRAEDTAVYYCARHLYGSSSYYVMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 645 bp, synthetic construct
  misc_feature 1..645: note = andibody
  source 1..645: mol_type = other DNA
  CDS 1..645: protein_id = 66; transl_table = 1; translation = DIQMTQSPSSLSASLGDRITITCHASQNINVWLSWYQQKPGKIP KLLVYKASNLHTGVPSRFSGSGSGTDFTLTISSLQPEDFATYYCQQGQSYPLTFGAGT KLEIKRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC
gacatccagatgacccagtctccatcctctctgtctgccagcctgggcgacagaatcaccatcacctgtcacgccagccagaacatcaacgtgtggctgtcctggtatcagcagaagcccggcaagatccccaagctgctggtgtacaaggcctccaatctgcacaccggcgtgccctctagattctccggatctggctctggcaccgactttaccctgacaatctccagcctgcagcctgaggacttcgccacctactattgtcagcagggccagagctaccctctgacctttggcgctggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 4 - protein, 214 aa, synthetic construct
  REGION 1..214: note = Synthetic Construct
DIQMTQSPSSLSASLGDRITITCHASQNINVWLSWYQQKPGKIPKLLVYKASNLHTGVPSRFSGSGSGTDFTLTISSLQPEDFATYYCQQGQSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1350 bp, synthetic construct
  misc_feature 1..1350: note = antibody
  source 1..1350: mol_type = other DNA
  CDS 1..1350: protein_id = 67; transl_table = 1; translation = EVQLVESGGGLVQPGGSLRLSCAASGFVFSSDDMSWVRQAPGKG LEWVAYISSGGDYTYYPDTVKGRFTISRDNAKNSLYLQMNSLRAEDTAVYYCARHLYG SSSYYVMDYWGQGTLVTVSSASTKGPSVFPLAPCSRSTSESTAALGCLVKDYFPEPVT VSWNSGALTSGVHTFPAVLQSSGLYSLSSVVTVPSSSLGTKTYTCNVDHKPSNTKVDK RVESKYGPPCPPCPAPEFLGGPSVFLFPPKPKDTLMISRTPEVTCVVVDVSQEDPEVQ FNWYVDGVEVHNAKTKPREEQFNSTYRVVSVLTVLHQDWLNGKEYKCKVSNKGLPSSI EKTISKAKGQPREPQVYTLPPSQEEMTKNQVSLTCLVKGFYPSDIAVEWESNGQPENN YKTTPPVLDSDGSFFLYSRLTVDKSRWQEGNVFSCSVMHEALHNHYTQKSLSLSLGK
gaggtgcagttggttgaatctggcggaggactggttcagcctggcggatctctgagactgtcttgtgccgcctctggcttcgtgttcagctccgacgatatgtcctgggtccgacaggctcctggcaaaggattggagtgggtcgcctacatctcttctggcggcgactacacctactatcccgacaccgtgaagggcagattcaccatctccagagacaacgccaagaactccctgtacctgcagatgaacagcctgagagccgaggacaccgccgtgtactactgtgccagacacctgtacggctcctccagctactacgtgatggactattggggccagggcaccctggtcacagtgtcctctgcttctaccaagggcccctccgtgttccctctggccccttgctccagatccacctccgagtctaccgccgctctgggctgcctcgtgaaggactacttccccgagcccgtgacagtgtcctggaactctggcgctctgacctccggcgtgcacacctttccagcagtgctgcagtcctccggcctgtactccctgtcctccgtcgtgactgtgccctccagctctctgggcaccaagacctacacctgtaacgtggaccacaagccctccaacaccaaggtggacaagagagtggaatctaagtacggccctccctgccccccttgtcctgcccctgaatttctgggcggacccagcgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactctcgcctgaccgtggacaagtcccggtggcaggaaggcaacgtgttctcctgctccgtgatgcacgaggccctgcacaaccactacacccagaagtccctgtctctgtccctgggcaagtga

SEQ ID NO: 6 - protein, 449 aa, synthetic construct
  REGION 1..449: note = Synthetic Construct
EVQLVESGGGLVQPGGSLRLSCAASGFVFSSDDMSWVRQAPGKGLEWVAYISSGGDYTYYPDTVKGRFTISRDNAKNSLYLQMNSLRAEDTAVYYCARHLYGSSSYYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 645 bp, synthetic construct; identical to SEQ ID NO: 3
  misc_feature 1..645: note = antibody
  source 1..645: mol_type = other DNA
  CDS 1..645: protein_id = 68; transl_table = 1; translation = DIQMTQSPSSLSASLGDRITITCHASQNINVWLSWYQQKPGKIP KLLVYKASNLHTGVPSRFSGSGSGTDFTLTISSLQPEDFATYYCQQGQSYPLTFGAGT KLEIKRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC

SEQ ID NO: 8 - protein, 214 aa, synthetic construct; identical to SEQ ID NO: 4
  REGION 1..214: note = Synthetic Construct

SEQ ID NO: 9 - DNA, 1347 bp, synthetic construct
  misc_feature 1..1347: note = antibody
  source 1..1347: mol_type = other DNA
  CDS 1..1347: protein_id = 69; transl_table = 1; translation = QVQLVQSGAEVKKPGASVKVSCKASGYTFTSYVIHWVRQAPGQG LEWMGYINPYHDVTKYNEKFQGRVTMTRDTSTSTVYMELSSLRSEDTAVYYCARQGDP YSGNYQFTYWGQGTLVTVSSASTKGPSVFPLAPCSRSTSESTAALGCLVKDYFPEPVT VSWNSGALTSGVHTFPAVLQSSGLYSLSSVVTVPSSNFGTQTYTCNVDHKPSNTKVDK TVERKCCVECPPCPAPPVAGPSVFLFPPKPKDTLMISRTPEVTCVVVDVSHEDPEVQF NWYVDGVEVHNAKTKPREEQFNSTFRVVSVLTVVHQDWLNGKEYKCKVSNKGLPAPIE KTISKTKGQPREPQVYTLPPSREEMTKNQVSLTCLVKGFYPSDIAVEWESNGQPENNY KTTPPMLDSDGSFFLYSKLTVDKSRWQQGNVFSCSVMHEALHNHYTQKSLSLSPGK
caggtgcagttggttcagtctggcgccgaagtgaagaaacctggcgcctctgtgaaggtgtcctgcaaggcttccggctacacctttaccagctacgtgatccactgggtccgacaggctccaggacaaggcttggagtggatgggctacatcaacccctaccacgacgtgaccaagtacaacgagaagttccagggcagagtgaccatgaccagagacacctccaccagcaccgtgtacatggaactgtccagcctgagatccgaggacaccgccgtgtactactgcgccagacagggcgacccttactccggcaactaccagtttacctactggggccagggcaccctggtcacagtttcttccgcttccaccaagggacccagcgttttccctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctctggcctgtactctctgtcctccgtcgtgaccgtgccttcctctaactttggcacccagacctacacctgtaatgtggaccacaagccttccaacaccaaggtggacaagaccgtggaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtctcacgaggatcccgaagtgcagttcaattggtacgtggacggcgtggaagtgcacaacgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgcctcgtgaagggcttctacccttccgatatcgccgtggaatgggagagcaatggccagcctgagaacaactacaagaccacacctcctatgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaatga

SEQ ID NO: 10 - protein, 448 aa, synthetic construct
  REGION 1..448: note = Synthetic Construct
QVQLVQSGAEVKKPGASVKVSCKASGYTFTSYVIHWVRQAPGQGLEWMGYINPYHDVTKYNEKFQGRVTMTRDTSTSTVYMELSSLRSEDTAVYYCARQGDPYSGNYQFTYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - DNA, 645 bp, synthetic construct
  misc_feature 1..645: note = antibody
  source 1..645: mol_type = other DNA
  CDS 1..645: protein_id = 70; transl_table = 1; translation = DIQMTQSPSSLSASVGDRVTITCRASQDVSTEVVWYQQKPGKAP KLLIYSASYRYSGVPSRFSGSGSGTDFTLTISSLQPEDFATYYCQQHYSTPYTFGQGT KLEIKRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC
gacatccagatgacccagtctccatcctctctgtccgcctctgtgggcgacagagtgaccatcacctgtagagcctctcaggacgtgtccaccgaggtcgtgtggtatcagcagaagcctggcaaggcccctaagctgctgatctactccgcctcctaccggtactctggcgtgccctctagattctccggctctggctctggcaccgactttaccctgacaatctccagcctgcagcctgaggacttcgccacctactactgccagcagcactacagcaccccttacacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 12 - protein, 214 aa, synthetic construct
  REGION 1..214: note = Synthetic Construct
DIQMTQSPSSLSASVGDRVTITCRASQDVSTEVVWYQQKPGKAPKLLIYSASYRYSGVPSRFSGSGSGTDFTLTISSLQPEDFATYYCQQHYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1350 bp, synthetic construct
  misc_feature 1..1350: note = andibody
  source 1..1350: mol_type = other DNA
  CDS 1..1350: protein_id = 71; transl_table = 1; translation = QVQLVQSGAEVKKPGASVKVSCKASGYTFTSYVIHWVRQAPGQG LEWMGYINPYHDVTKYNEKFQGRVTMTRDTSTSTVYMELSSLRSEDTAVYYCARQGDP YSGNYQFTYWGQGTLVTVSSASTKGPSVFPLAPCSRSTSESTAALGCLVKDYFPEPVT VSWNSGALTSGVHTFPAVLQSSGLYSLSSVVTVPSSSLGTKTYTCNVDHKPSNTKVDK RVESKYGPPCPPCPAPEFLGGPSVFLFPPKPKDTLMISRTPEVTCVVVDVSQEDPEVQ FNWYVDGVEVHNAKTKPREEQFNSTYRVVSVLTVLHQDWLNGKEYKCKVSNKGLPSSI EKTISKAKGQPREPQVYTLPPSQEEMTKNQVSLTCLVKGFYPSDIAVEWESNGQPENN YKTTPPVLDSDGSFFLYSRLTVDKSRWQEGNVFSCSVMHEALHNHYTQKSLSLSLGK
caggtgcagttggttcagtctggcgccgaagtgaagaaacctggcgcctctgtgaaggtgtcctgcaaggcttccggctacacctttaccagctacgtgatccactgggtccgacaggctccaggacaaggcttggagtggatgggctacatcaacccctaccacgacgtgaccaagtacaacgagaagttccagggcagagtgaccatgaccagagacacctccaccagcaccgtgtacatggaactgtccagcctgagatccgaggacaccgccgtgtactactgcgccagacagggcgacccttactccggcaactaccagtttacctactggggccagggcaccctggtcacagtttcttccgcttctaccaagggcccctccgtgttccctctggccccttgctccagatccacctccgagtctaccgccgctctgggctgcctcgtgaaggactacttccccgagcccgtgacagtgtcctggaactctggcgctctgacctccggcgtgcacacctttccagcagtgctgcagtcctccggcctgtactccctgtcctccgtcgtgactgtgccctccagctctctgggcaccaagacctacacctgtaacgtggaccacaagccctccaacaccaaggtggacaagagagtggaatctaagtacggccctccctgccccccttgtcctgcccctgaatttctgggcggacccagcgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactctcgcctgaccgtggacaagtcccggtggcaggaaggcaacgtgttctcctgctccgtgatgcacgaggccctgcacaaccactacacccagaagtccctgtctctgtccctgggcaagtga

SEQ ID NO: 14 - protein, 449 aa, synthetic construct
  REGION 1..449: note = Synthetic Construct
QVQLVQSGAEVKKPGASVKVSCKASGYTFTSYVIHWVRQAPGQGLEWMGYINPYHDVTKYNEKFQGRVTMTRDTSTSTVYMELSSLRSEDTAVYYCARQGDPYSGNYQFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - DNA, 645 bp, synthetic construct; identical to SEQ ID NO: 11
  misc_feature 1..645: note = antibody
  source 1..645: mol_type = other DNA
  CDS 1..645: protein_id = 72; transl_table = 1; translation = DIQMTQSPSSLSASVGDRVTITCRASQDVSTEVVWYQQKPGKAP KLLIYSASYRYSGVPSRFSGSGSGTDFTLTISSLQPEDFATYYCQQHYSTPYTFGQGT KLEIKRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC

SEQ ID NO: 16 - protein, 214 aa, synthetic construct; identical to SEQ ID NO: 12
  REGION 1..214: note = Synthetic Construct

SEQ ID NO: 17 - DNA, 366 bp, synthetic construct
  misc_feature 1..366: note = antibody variable region
  source 1..366: mol_type = other DNA
  CDS 1..366: protein_id = 73; transl_table = 1; translation = EVQLVESGGGLVQPGGSLRLSCAASGFVFSSDDMSWVRQAPGKG LEWVAYISSGGDYTYYPDTVKGRFTISRDNAKNSLYLQMNSLRAEDTAVYYCARHLYG SSSYYVMDYWGQGTLVTVSS
gaggtgcagttggttgaatctggcggaggactggttcagcctggcggatctctgagactgtcttgtgccgcctctggcttcgtgttcagctccgacgatatgtcctgggtccgacaggctcctggcaaaggattggagtgggtcgcctacatctcttctggcggcgactacacctactatcccgacaccgtgaagggcagattcaccatctccagagacaacgccaagaactccctgtacctgcagatgaacagcctgagagccgaggacaccgccgtgtactactgtgccagacacctgtacggctcctccagctactacgtgatggactattggggccagggcaccctggtcacagtgtcctct

SEQ ID NO: 18 - protein, 122 aa, synthetic construct
  REGION 1..122: note = Synthetic Construct
EVQLVESGGGLVQPGGSLRLSCAASGFVFSSDDMSWVRQAPGKGLEWVAYISSGGDYTYYPDTVKGRFTISRDNAKNSLYLQMNSLRAEDTAVYYCARHLYGSSSYYVMDYWGQGTLVTVSS

SEQ ID NO: 19 - DNA, 321 bp, synthetic construct
  misc_feature 1..321: note = antibody variable region
  source 1..321: mol_type = other DNA
  CDS 1..321: protein_id = 74; transl_table = 1; translation = DIQMTQSPSSLSASLGDRITITCHASQNINVWLSWYQQKPGKIP KLLVYKASNLHTGVPSRFSGSGSGTDFTLTISSLQPEDFATYYCQQGQSYPLTFGAGT KLEIK
gacatccagatgacccagtctccatcctctctgtctgccagcctgggcgacagaatcaccatcacctgtcacgccagccagaacatcaacgtgtggctgtcctggtatcagcagaagcccggcaagatccccaagctgctggtgtacaaggcctccaatctgcacaccggcgtgccctctagattctccggatctggctctggcaccgactttaccctgacaatctccagcctgcagcctgaggacttcgccacctactattgtcagcagggccagagctaccctctgacctttggcgctggcaccaagctggaaatcaag

SEQ ID NO: 20 - protein, 107 aa, synthetic construct
  REGION 1..107: note = Synthetic Construct
DIQMTQSPSSLSASLGDRITITCHASQNINVWLSWYQQKPGKIPKLLVYKASNLHTGVPSRFSGSGSGTDFTLTISSLQPEDFATYYCQQGQSYPLTFGAGTKLEIK

SEQ ID NO: 21 - DNA, 366 bp, synthetic construct
  misc_feature 1..366: note = antibody variable region
  source 1..366: mol_type = other DNA
  CDS 1..366: protein_id = 75; transl_table = 1; translation = QVQLVQSGAEVKKPGASVKVSCKASGYTFTSYVIHWVRQAPGQG LEWMGYINPYHDVTKYNEKFQGRVTMTRDTSTSTVYMELSSLRSEDTAVYYCARQGDP YSGNYQFTYWGQGTLVTVSS
caggtgcagttggttcagtctggcgccgaagtgaagaaacctggcgcctctgtgaaggtgtcctgcaaggcttccggctacacctttaccagctacgtgatccactgggtccgacaggctccaggacaaggcttggagtggatgggctacatcaacccctaccacgacgtgaccaagtacaacgagaagttccagggcagagtgaccatgaccagagacacctccaccagcaccgtgtacatggaactgtccagcctgagatccgaggacaccgccgtgtactactgcgccagacagggcgacccttactccggcaactaccagtttacctactggggccagggcaccctggtcacagtttcttcc

SEQ ID NO: 22 - protein, 122 aa, synthetic construct
  REGION 1..122: note = Synthetic Construct
QVQLVQSGAEVKKPGASVKVSCKASGYTFTSYVIHWVRQAPGQGLEWMGYINPYHDVTKYNEKFQGRVTMTRDTSTSTVYMELSSLRSEDTAVYYCARQGDPYSGNYQFTYWGQGTLVTVSS

SEQ ID NO: 23 - DNA, 321 bp, synthetic construct
  misc_feature 1..321: note = antibody variable region
  source 1..321: mol_type = other DNA
  CDS 1..321: protein_id = 76; transl_table = 1; translation = DIQMTQSPSSLSASVGDRVTITCRASQDVSTEVVWYQQKPGKAP KLLIYSASYRYSGVPSRFSGSGSGTDFTLTISSLQPEDFATYYCQQHYSTPYTFGQGT KLEIK
gacatccagatgacccagtctccatcctctctgtccgcctctgtgggcgacagagtgaccatcacctgtagagcctctcaggacgtgtccaccgaggtcgtgtggtatcagcagaagcctggcaaggcccctaagctgctgatctactccgcctcctaccggtactctggcgtgccctctagattctccggctctggctctggcaccgactttaccctgacaatctccagcctgcagcctgaggacttcgccacctactactgccagcagcactacagcaccccttacacctttggccagggcaccaagctggaaatcaag

SEQ ID NO: 24 - protein, 107 aa, synthetic construct
  REGION 1..107: note = Synthetic Construct
DIQMTQSPSSLSASVGDRVTITCRASQDVSTEVVWYQQKPGKAPKLLIYSASYRYSGVPSRFSGSGSGTDFTLTISSLQPEDFATYYCQQHYSTPYTFGQGTKLEIK

SEQ ID NO: 25 - protein, 5 aa, synthetic construct
  REGION 1..5: note = CDR
SDDMS

SEQ ID NO: 26 - protein, 17 aa, synthetic construct
  REGION 1..17: note = CDR
YISSGGDYTYYPDTVKG

SEQ ID NO: 27 - protein, 13 aa, synthetic construct
  REGION 1..13: note = CDR
HLYGSSSYYVMDY

SEQ ID NO: 28 - protein, 11 aa, synthetic construct
  REGION 1..11: note = CDR
HASQNINVWLS

SEQ ID NO: 29 - protein, 7 aa, synthetic construct
  REGION 1..7: note = CDR
KASNLHT

SEQ ID NO: 30 - protein, 9 aa, synthetic construct
  REGION 1..9: note = CDR
QQGQSYPLT

SEQ ID NO: 31 - protein, 5 aa, synthetic construct
  REGION 1..5: note = CDR
SYVIH

SEQ ID NO: 32 - protein, 17 aa, synthetic construct
  REGION 1..17: note = CDR
YINPYHDVTKYNEKFQG

SEQ ID NO: 33 - protein, 13 aa, synthetic construct
  REGION 1..13: note = CDR
QGDPYSGNYQFTY

SEQ ID NO: 34 - protein, 11 aa, synthetic construct
  REGION 1..11: note = CDR
RASQDVSTEVV

SEQ ID NO: 35 - protein, 7 aa, synthetic construct
  REGION 1..7: note = CDR
SASYRYS

SEQ ID NO: 36 - protein, 9 aa, synthetic construct
  REGION 1..9: note = CDR
QQHYSTPYT

SEQ ID NO: 37 - DNA, 1359 bp, synthetic construct
  misc_feature 1..1359: note = heavy chain of chimera anti-CD80 antibody
  source 1..1359: mol_type = other DNA
gaagtgaagctgttggagtctggcggcggactggttaagcctggcggatctctgaagctgtcttgtgccgcctctggcttcgtgttcagctccgacgatatgtcctgggtccgacagtctcctgagaagcggcttgagtgggtcgcctacatctcttccggcggagactacacctactatcccgacaccgtgaagggcagattcaccatctccagagacaacgccaagaacaccctgtacctgcagatgtccagcctgaagtctgaggacaccgccatgtactactgcgccagacacctgtacggctcctccagctactacgtgatggactattggggccagggcacctctgtcacagtgtcctctgcttccaccaagggacccagcgtgttccctctggctccttccagcaagtctacctctggcggaacagctgctctgggctgcctggtcaaggactactttcctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtcccacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgagaaccccaggtttacaccttgcctccatctcgggacgagctgaccaagaaccaggtgtccctgacctgtctcgtgaagggcttctacccctccgacattgccgtggaatgggagtctaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgagccccggcaaatga

SEQ ID NO: 38 - protein, 452 aa, synthetic construct
  REGION 1..452: note = heavy chain of chimera anti-CD80 antibody
EVKLLESGGGLVKPGGSLKLSCAASGFVFSSDDMSWVRQSPEKRLEWVAYISSGGDYTYYPDTVKGRFTISRDNAKNTLYLQMSSLKSEDTAMYYCARHLYGSSSYYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 645 bp, synthetic construct
  misc_feature 1..645: note = light chain of chimera anti-CD80 antibody
  source 1..645: mol_type = other DNA
gacatccagatgacccagtctccatcctctctgtctgccagcctgggcgacaccatcaccatcacatgtcacgccagccagaacatcaacgtgtggctgtcctggtatcagcagaagcccggcaacatccccaagctgctggtgtacaaggcctccaatctgcacaccggcgtgccctctagattctccggatctggctctggcaccggctttaccctgacaatcagctccttgcagcccgaggacattgccacctactactgtcagcagggccagagctaccctctgacctttggcgctggcaccaagctggaactgaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 40 - protein, 214 aa, synthetic construct
  REGION 1..214: note = light chain of chimera anti-CD80 antibody
DIQMTQSPSSLSASLGDTITITCHASQNINVWLSWYQQKPGNIPKLLVYKASNLHTGVPSRFSGSGSGTGFTLTISSLQPEDIATYYCQQGQSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 1359 bp, synthetic construct
  misc_feature 1..1359: note = heavy chain of chimera anti-CD86 antibody
  source 1..1359: mol_type = other DNA
gaggttcagttgcagcagtctggacccgagctggtcaagcctggcacctccgtgaagatgtcctgcaaggcctccggctacaccttcaccagctacgtgatccactgggtcaaagagaagccaggccagggccttgagtggatcggctacatcaacccctaccacgacgtgaccaagtacaacgagaagttcaagggcagagctaccctgacctccgacaagtcctcttccaccgcctacatggaactgtccagcctgacctctgaggactccgccgtgtactactgctctagacagggcgacccttactccggcaactaccagtttacctactggggccagggcaccctggtcacagtttctgccgcttccaccaagggacccagcgtgttccctctggctccttccagcaagtctacctctggcggaacagctgctctgggctgcctggtcaaggactactttcctgagcctgtgaccgtgtcctggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtcccacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgagaaccccaggtttacaccttgcctccatctcgggacgagctgaccaagaaccaggtgtccctgacctgtctcgtgaagggcttctacccctccgacattgccgtggaatgggagtctaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgagccccggcaaatga

SEQ ID NO: 42 - protein, 452 aa, synthetic construct
  REGION 1..452: note = heavy chain of chimera anti-CD86 antibody
EVQLQQSGPELVKPGTSVKMSCKASGYTFTSYVIHWVKEKPGQGLEWIGYINPYHDVTKYNEKFKGRATLTSDKSSSTAYMELSSLTSEDSAVYYCSRQGDPYSGNYQFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 645 bp, synthetic construct
  misc_feature 1..645: note = light chain of chimera anti-CD86 antibody
  source 1..645: mol_type = other DNA
gacatcgtgatgtcccagagccacaagttcatgtccacctccgtgggcgacagagtgtctatcgcctgcaaggcctctcaggacgtgtccacagaggtcgtgtggttccagcagaagcctggccagtctcctaagctgctgatctactccgcctcctacagatacaccggcgtgcccgatagattcaccggctctggctctggcaccgactttaccttcaccatcagctccgtgcaggccgaggatctggccgtgtactactgtcagcagcactacagcaccccttacacctttggcggaggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 44 - protein, 214 aa, synthetic construct
  REGION 1..214: note = light chain of chimera anti-CD86 antibody
DIVMSQSHKFMSTSVGDRVSIACKASQDVSTEVVWFQQKPGQSPKLLIYSASYRYTGVPDRFTGSGSGTDFTFTISSVQAEDLAVYYCQQHYSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 366 bp, synthetic construct
  misc_feature 1..366: note = heavy chain variable region of chimera anti-CD80 antibody
  source 1..366: mol_type = other DNA
gaagtgaagctgttggagtctggcggcggactggttaagcctggcggatctctgaagctgtcttgtgccgcctctggcttcgtgttcagctccgacgatatgtcctgggtccgacagtctcctgagaagcggcttgagtgggtcgcctacatctcttccggcggagactacacctactatcccgacaccgtgaagggcagattcaccatctccagagacaacgccaagaacaccctgtacctgcagatgtccagcctgaagtctgaggacaccgccatgtactactgcgccagacacctgtacggctcctccagctactacgtgatggactattggggccagggcacctctgtcacagtgtcctct

SEQ ID NO: 46 - protein, 122 aa, synthetic construct
  REGION 1..122: note = heavy chain variable region of chimera anti-CD80 antibody
EVKLLESGGGLVKPGGSLKLSCAASGFVFSSDDMSWVRQSPEKRLEWVAYISSGGDYTYYPDTVKGRFTISRDNAKNTLYLQMSSLKSEDTAMYYCARHLYGSSSYYVMDYWGQGTSVTVSS

SEQ ID NO: 47 - DNA, 321 bp, synthetic construct
  misc_feature 1..321: note = light chain variable region of chimera anti-CD80 antibody
  source 1..321: mol_type = other DNA
gacatccagatgacccagtctccatcctctctgtctgccagcctgggcgacaccatcaccatcacatgtcacgccagccagaacatcaacgtgtggctgtcctggtatcagcagaagcccggcaacatccccaagctgctggtgtacaaggcctccaatctgcacaccggcgtgccctctagattctccggatctggctctggcaccggctttaccctgacaatcagctccttgcagcccgaggacattgccacctactactgtcagcagggccagagctaccctctgacctttggcgctggcaccaagctggaactgaag

SEQ ID NO: 48 - protein, 107 aa, synthetic construct
  REGION 1..107: note = light chain variable region of chimera anti-CD80 antibody
DIQMTQSPSSLSASLGDTITITCHASQNINVWLSWYQQKPGNIPKLLVYKASNLHTGVPSRFSGSGSGTGFTLTISSLQPEDIATYYCQQGQSYPLTFGAGTKLELK

SEQ ID NO: 49 - DNA, 366 bp, synthetic construct
  misc_feature 1..366: note = heavy chain variable region of chimera anti-CD86 antibody
  source 1..366: mol_type = other DNA
gaggttcagttgcagcagtctggacccgagctggtcaagcctggcacctccgtgaagatgtcctgcaaggcctccggctacaccttcaccagctacgtgatccactgggtcaaagagaagccaggccagggccttgagtggatcggctacatcaacccctaccacgacgtgaccaagtacaacgagaagttcaagggcagagctaccctgacctccgacaagtcctcttccaccgcctacatggaactgtccagcctgacctctgaggactccgccgtgtactactgctctagacagggcgacccttactccggcaactaccagtttacctactggggccagggcaccctggtcacagtttctgcc

SEQ ID NO: 50 - protein, 122 aa, synthetic construct
  REGION 1..122: note = heavy chain variable region of chimera anti-CD86 antibody
EVQLQQSGPELVKPGTSVKMSCKASGYTFTSYVIHWVKEKPGQGLEWIGYINPYHDVTKYNEKFKGRATLTSDKSSSTAYMELSSLTSEDSAVYYCSRQGDPYSGNYQFTYWGQGTLVTVSA

SEQ ID NO: 51 - DNA, 321 bp, synthetic construct
  misc_feature 1..321: note = light chain variable region of chimera anti-CD86 antibody
  source 1..321: mol_type = other DNA
gacatcgtgatgtcccagagccacaagttcatgtccacctccgtgggcgacagagtgtctatcgcctgcaaggcctctcaggacgtgtccacagaggtcgtgtggttccagcagaagcctggccagtctcctaagctgctgatctactccgcctcctacagatacaccggcgtgcccgatagattcaccggctctggctctggcaccgactttaccttcaccatcagctccgtgcaggccgaggatctggccgtgtactactgtcagcagcactacagcaccccttacacctttggcggaggcaccaagctggaaatcaag

SEQ ID NO: 52 - protein, 107 aa, synthetic construct
  REGION 1..107: note = light chain variable region of chimera anti-CD86 antibody
DIVMSQSHKFMSTSVGDRVSIACKASQDVSTEVVWFQQKPGQSPKLLIYSASYRYTGVPDRFTGSGSGTDFTFTISSVQAEDLAVYYCQQHYSTPYTFGGGTKLEIK

SEQ ID NO: 53 - protein, 5 aa, synthetic construct
  REGION 1..5: note = CDRH1 of chimera anti-CD80 antibody
SDDMS

SEQ ID NO: 54 - protein, 17 aa, synthetic construct
  REGION 1..17: note = CDRH2 of chimera anti-CD80 antibody
YISSGGDYTYYPDTVKG

SEQ ID NO: 55 - protein, 13 aa, synthetic construct
  REGION 1..13: note = CDRH3 of chimera anti-CD80 antibody
HLYGSSSYYVMDY

SEQ ID NO: 56 - protein, 11 aa, synthetic construct
  REGION 1..11: note = CDRL1 of chimera anti-CD80 antibody
HASQNINVWLS

SEQ ID NO: 57 - protein, 7 aa, synthetic construct
  REGION 1..7: note = CDRL2 of chimera anti-CD80 antibody
KASNLHT

SEQ ID NO: 58 - protein, 9 aa, synthetic construct
  REGION 1..9: note = CDRL3 of chimera anti-CD80 antibody
QQGQSYPLT

SEQ ID NO: 59 - protein, 5 aa, synthetic construct
  REGION 1..5: note = CDRH1 of chimera anti-CD86 antibody
SYVIH

SEQ ID NO: 60 - protein, 17 aa, synthetic construct
  REGION 1..17: note = CDRH2 of chimera anti-CD86 antibody
YINPYHDVTKYNEKFKG

SEQ ID NO: 61 - protein, 13 aa, synthetic construct
  REGION 1..13: note = CDRH3 of chimera anti-CD86 antibody
QGDPYSGNYQFTY

SEQ ID NO: 62 - protein, 11 aa, synthetic construct
  REGION 1..11: note = CDRL1 of chimera anti-CD86 antibody
KASQDVSTEVV

SEQ ID NO: 63 - protein, 7 aa, synthetic construct
  REGION 1..7: note = CDRL2 of chimera anti-CD86 antibody
SASYRYT

SEQ ID NO: 64 - protein, 9 aa, synthetic construct
  REGION 1..9: note = CDRL3 of chimera anti-CD86 antibody
QQHYSTPYT

SEQ ID NO: 65 - protein, 448 aa, synthetic construct; identical to SEQ ID NO: 2

SEQ ID NO: 66 - protein, 214 aa, synthetic construct; identical to SEQ ID NO: 4

SEQ ID NO: 67 - protein, 449 aa, synthetic construct; identical to SEQ ID NO: 6

SEQ ID NO: 68 - protein, 214 aa, synthetic construct; identical to SEQ ID NO: 4

SEQ ID NO: 69 - protein, 448 aa, synthetic construct; identical to SEQ ID NO: 10

SEQ ID NO: 70 - protein, 214 aa, synthetic construct; identical to SEQ ID NO: 12

SEQ ID NO: 71 - protein, 449 aa, synthetic construct; identical to SEQ ID NO: 14

SEQ ID NO: 72 - protein, 214 aa, synthetic construct; identical to SEQ ID NO: 12

SEQ ID NO: 73 - protein, 122 aa, synthetic construct; identical to SEQ ID NO: 18

SEQ ID NO: 74 - protein, 107 aa, synthetic construct; identical to SEQ ID NO: 20

SEQ ID NO: 75 - protein, 122 aa, synthetic construct; identical to SEQ ID NO: 22

SEQ ID NO: 76 - protein, 107 aa, synthetic construct; identical to SEQ ID NO: 24

(19) United States
(12) Patent Application Publication
(10) Pub. No.: US 2025/0101110 A1
(43) Pub. Date: Mar. 27, 2025

(54) ANTIBODY INDUCING IMMUNE TOLERANCE, INDUCED LYMPHOCYTE, AND CELL THERAPY AGENT THERAPEUTIC METHOD USING INDUCED LYMPHOCYTE

(71) Applicant: Junten Bio Co., Ltd., Tokyo (JP)

(72) Inventors: Ryu Maeda, Sapporo-shi (JP); Masayuki Kawakami, Sapporo-shi (JP); Koichiro Uchida, Tokyo (JP); Kazuyoshi Takeda, Tokyo (JP); Ko Okumura, Tokyo (JP)

(21) Appl. No.: 18/920,129

(22) Filed: Oct. 18, 2024

Related U.S. Application Data
(63) Continuation of application No. 17/254,006, filed on Dec. 18, 2020, now Pat. No. 12,139,537.

(30) Foreign Application Priority Data
Jun. 22, 2018 (JP) 2018-118996

(51) Int. Cl. C07K 16/28; A61K 35/17; A61K 39/00; A61P 37/06; C12N 5/0783

(52) U.S. Cl. CPC C07K 16/2827; A61K 35/17; A61K 40/11; A61K 40/22; A61K 40/418; A61P 37/06; C12N 5/0636; A61K 2039/505; C07K 2317/24; C07K 2317/52; C07K 2317/565; C07K 2317/76; C12N 2501/998

(57) ABSTRACT

The present disclosure provides an antibody for inducing immune tolerance, an induced lymphocyte, and a cell therapy agent therapeutic method using the induced lymphocyte. Specifically, the present disclosure provides an antibody that inhibits the interaction between CD80 and/or CD86 expressed on the surface of a certain cell and CD28 expressed on the surface of another cell, and substantially does not induce immune activation-induced cytokines. In a specific embodiment, the Fc portion of the antibody substantially does not produce the immune activation-induced cytokines.

### TECHNICAL FIELD

The present disclosure relates to a novel technology related to immune tolerance. More specifically, the present disclosure relates to an antibody that induces immune tolerance, induced lymphocyte, or cell therapy agent/therapeutic method using an induced lymphocyte.

### BACKGROUND ART

Liver transplantation has been widely used as the final treatment on terminal liver failure patients. 20,000 or more liver transplantations are performed abroad, and more than 500 are performed in Japan annually.

Transplantation is one of the primary treatments chosen for terminal organ failure of the kidney, heart, liver, pancreas, or the like. Despite the dramatic advancement in the treatment of graft rejection in recent years, the majority of transplantations are ultimately rejected without any immunosuppressive regimen. Today's drug immunosuppressive regimen which is dependent on continuous drug therapy suppresses not only responses that are clearly directed to transplantation, but also all immune responses, such that organ transplant patients become more vulnerable to increased sensitivity to infections and cancer.

While regenerative medicine has also drawn attention, immune rejection can ultimately occur without any autologous cells even if induced pluripotent stem cells (iPS cells) or the like are used. Thus, immune tolerance technologies have garnered attention.

Such technologies for inducing immune tolerance include induction of an antigen specific non-immune response (anergy) of T cells. Specific technologies reported include a technology for directly administering an antibody which inhibits interactions between CD80/CD86 on antigen presenting cells and CD28 on unactivated (naïve) T cells to an organ transplant patient to induce donor antigen specific anergy in the body (Patent Literature 1) and a technology of co-culturing recipient cells and radiation irradiated donor cells in the presence of the same antibody to induce donor antigen specific anergic cells ex vivo and returning said cells to the recipient (Patent Literature 2, Patent Literature 3, and Non Patent Literatures 1 to 3)

### CITATION LIST

### Patent Literature

[PTL 1] Japanese National Phase PCT Laid-open Publication No. 2002-504120
[PTL 2] Japanese National Phase PCT Laid-open Publication No. 2007-131598
[PTL 3] Japanese National Phase PCT Laid-open Publication No. 2016-520081

### Non Patent Literature

[NPL 1] Satoru Todo et al. Hepatorogy, 64 (vol.2), 632-643 (2016)
[NPL 2] Teraoka S, Koyama I, Bashuda H, Uchida K, Tonsho M, et al. (2017) J Transplant Res 2(1) p1-8
[NPL 3] Bashuda H et al., J. Clin. Invest. 115:1896-1902 (2005).

### SUMMARY OF INVENTION

### Solution to Problem

The inventors attempted to induce a non-immune response (anergy) using an antibody with a specific human Fc region that inhibits the interaction between CD80/CD86 and CD28, resulting in discovering that binding to a cell involved in the immune system such as a macrophage, neutrophil, or natural killer (NK) cell activates these cells and induces the release of a humoral factor such as interleukin or interferon (IFN) that is responsible for immune responses. The inventors newly discovered that release of these humoral factors induces disadvantageous activation of the immune system that is the opposite of immune tolerance, so that the effect of immune tolerance can be improved, or the attenuation of the effect of immune tolerance can be suppressed by adjusting the induction of non-immune responses (anergy) using an antibody with a human Fc region that does not bind to these cells. The inventors thereby provide an antibody with a structure having a feature of improving, or not attenuating, the effect of immune tolerance.

Therefore, the present disclosure provides the following.
(1) An antibody or variant thereof that inhibits an interaction between CD80 and/or CD86 expressed on a surface of a cell, and CD28 expressed on a surface of another cell, antibody or wherein the variant thereof does not substantially induce the production of a cytokine by immunostimulation.
(2) The antibody or variant thereof of the preceding item, wherein the cytokine comprises interferon γ.
(3) The antibody or variant thereof of any of the preceding items, which is a chimeric antibody.
(4) The antibody or variant thereof of any one of the preceding items, wherein a subclass of the antibody is IgG1.
(5) The antibody or variant thereof of any of the preceding items, having a heavy chain with the amino acid sequence set forth in SEQ ID NO: 38 or 42, and a light chain with the amino acid sequence set forth in SEQ ID NO: 40 or 44.
(6) The antibody or variant thereof of any of the preceding items, comprising:
   (a) a VH comprising CDRH1 of the amino acid sequence set forth in SEQ ID NO: 53, CDRH2 of the amino acid sequence set forth in SEQ ID NO: 54, and CDRH3 of the amino acid sequence set forth in SEQ ID NO: 55, and a VL comprising CDRL1 of the amino acid sequence set forth in SEQ ID NO: 56, CDRL2 of the amino acid sequence set forth in SEQ ID NO: 57, and CDRL3 set forth in SEQ ID NO: 58; or
   (b) a VH comprising CDRH1 of the amino acid sequence set forth in SEQ ID NO: 59, CDRH2 of the amino acid sequence set forth in SEQ ID NO: 60, and CDRH3 of the amino acid sequence set forth in SEQ ID NO: 61, and a VL comprising CDRL1 of the amino acid sequence set forth in SEQ ID NO: 62, CDRL2 of the amino acid sequence set forth in SEQ ID NO: 63, and CDRL3 set forth in SEQ ID NO: 64.
(7) The antibody or variant thereof of any of the preceding items, comprising:
   (a) a VH having the amino acid sequence set forth in SEQ ID NO: 46 or a variant sequence thereof, and a VL having the amino acid sequence set forth in SEQ ID NO: 48 or a variant sequence thereof; or
   (b) a VH having the amino acid sequence set forth in SEQ ID NO: 50 or a variant sequence thereof, and a VL having the amino acid sequence set forth in SEQ ID NO: 52 or a variant sequence thereof.
(8) The antibody or variant thereof of any of the preceding items, wherein an Fc moiety of the antibody is a moiety that does not substantially induce the production of a cytokine by immunostimulation.
(9) The antibody or variant thereof of any of the preceding items, wherein a subclass of the antibody is IgG2 or IgG4.
(10) The antibody or variant thereof of any of the preceding items, wherein a subclass of the antibody is IgG4.
(11) The antibody or variant thereof of any of the preceding items, wherein the variant of the antibody is a variant lacking an Fc moiety of the antibody.
(12) The antibody or variant thereof of any of the preceding items, wherein the variant of the antibody is an Fab antibody, an F(ab′)₂ antibody, an Fab′ antibody, an Fv antibody, or an scFv antibody.
(13) The antibody or variant thereof of any of the preceding items, having an ability to induce immune tolerance.
(14) The antibody or variant thereof of any of the preceding items, wherein the cell expressing CD80 and/or CD86 is an antigen presenting cell, and the another cell expressing CD28 is a T cell.
(15) The antibody of any of the preceding items, wherein the antibody or variant thereof is an antagonistic anti-CD80 anti-CD86 an antibody, an antagonistic antibody, antagonistic anti-CD28 antibody, or an antagonistic bispecific antibody to CD80 and CD86, or a variant thereof.
(16) The antibody or variant thereof of any of the preceding items, which is a humanized antibody or a human antibody, or a variant thereof.
(17) The antibody or variant thereof of any of the preceding items, comprising an Fc moiety of IgG2 or IgG4 of a human antibody.
(18) The antibody or variant thereof of any of the preceding items, comprising an Fc moiety of IgG4 of a human antibody.
(19) The antibody or variant thereof of any of the preceding items, comprising:
   (a) a VH comprising CDRH1 of the amino acid sequence set forth in SEQ ID NO: 25, CDRH2 of the amino acid sequence set forth in SEQ ID NO: 26, and CDRH3 of the amino acid sequence set forth in SEQ ID NO: 27, and a VL comprising CDRL1 of the amino acid sequence set forth in SEQ ID NO: 28, CDRL2 of the amino acid sequence set forth in SEQ ID NO: 29, and CDRL3 set forth in SEQ ID NO: 30; or
   (b) a VH comprising CDRH1 of the amino acid sequence set forth in SEQ ID NO: 31, CDRH2 of the amino acid sequence set forth in SEQ ID NO: 32, and CDRH3 of the amino acid sequence set forth in SEQ ID NO: 33, and a VL comprising CDRL1 of the amino acid sequence set forth in SEQ ID NO: 34, CDRL2 of the amino acid sequence set forth in SEQ ID NO: 35, and CDRL3 set forth in SEQ ID NO 36.
(20) The antibody or variant thereof of any of the preceding items, comprising:
   (a) a VH having the amino acid sequence set forth in SEQ ID NO: 18 or a variant sequence thereof, and a VL having the amino acid sequence set forth in SEQ ID NO: 20 or a variant sequence thereof; or
   (b) a VH having the amino acid sequence set forth in SEQ ID NO: 22 or a variant sequence thereof, and a VL having the amino acid sequence set forth in SEQ ID NO: 24 or a variant sequence thereof.
(21) A nucleic acid molecule encoding the amino acid sequence or a portion thereof of the antibody or variant thereof of any of the preceding items.
(22) The nucleic acid molecule of any of the preceding items, comprising:
   (a) a polynucleotide encoding a VH, having the nucleotide sequence set forth in SEQ ID NO: 17 or SEQ ID NO: 45, and a polynucleotide encoding a VL, having the nucleotide sequence set forth in SEQ ID NO: 19 or SEQ ID NO: 47; or
   (b) a polynucleotide encoding a VH, having the nucleotide sequence set forth in SEQ ID NO: 21 or SEQ ID NO: 49, and a polynucleotide encoding a VL, having the nucleotide sequence
(23) A vector having the nucleic acid molecule of any of the preceding items.
(24) A cell comprising the nucleic acid molecule of any of the preceding items or the vector of item 23.
(25) A manufacturing method of the antibody or variant thereof of any of the preceding items, comprising culturing the cell of any of the preceding items.
(26) A cell having immune tolerance induced with the antibody or variant thereof of any of the preceding items.
(27) The cell of any of the preceding items, wherein the immune tolerance is induced by mixing the antibody or variant thereof, a cell derived from a subject, and an antigen that is not derived from the subject or a material containing the antigen.
(28) A composition for preparing a cell having immune tolerance induced, comprising at least one of the antibodies or variants thereof of any of the preceding items.
(29) The composition of any of the preceding items, comprising an antagonistic anti-CD80 antibody, an antagonistic anti-CD86 antibody, an antagonistic anti-CD28 antibody, or an antagonistic bispecific antibody to CD80 and CD86, or a variant thereof, or any combination thereof.
(30) A method for manufacturing a cell for treating or preventing a disease, disorder, or condition caused by an antigen that is not derived from a subject, comprising mixing the antibody or variant thereof of any of the preceding items, a cell derived from the subject, and an antigen that is not derived from the subject or a material containing the antigen.
(31) The method of any of the preceding items, wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(32) The method of any of the preceding items, wherein a material containing the antigen is a cell.
(33) A cell manufactured by the method of any of the preceding items.
(34) The cell of any of the preceding items, comprising a T cell.
(35) A cell therapy agent comprising a cell having immune tolerance induced with the antibody or variant thereof of any of the preceding items, a cell manufactured by the method of any of the preceding items, or the cell of any of the preceding items.
(36) A composition for treating or preventing a disease, disorder, or condition caused by an antigen that is not derived from a subject, comprising a cell having immune tolerance induced with the antibody or variant thereof of any of the preceding items, a cell manufactured by the method of any of the preceding items, or the cell of any of the preceding items.
(37) The composition of any of the preceding items, wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(38) The composition of any of the preceding items, wherein the rejection is characterized in that a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a cornea, an eye ball, or a bone marrow.
(39) A method for treating or preventing a disease, disorder, or condition caused by an antigen that is not derived from a subject in the subject, comprising administering to the subject an effective amount of a cell having immune tolerance induced with the antibody or variant thereof of any of the preceding items, a cell manufactured by the method of any of the preceding items, or the cell of any of the preceding items.
(40) The method of any of the preceding items, wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(41) The method of any of the preceding items, wherein the rejection is characterized in that a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a cornea, an eye ball, or a bone marrow.
(39A) Use of a cell having immune tolerance induced with the antibody or variant thereof of any of the preceding items, a cell manufactured by the method of any of the preceding items, or the cell of any of the preceding items, for the manufacture of a medicament for treating or preventing a disease, disorder, or condition caused by an antigen that is not derived from a subject in the subject.
(40A) The use of any of the preceding items, wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(41A) The use of any of the preceding items, wherein the rejection is characterized in that a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a cornea, an eye ball, or a bone marrow.
(39B) A cell having immune tolerance induced with the antibody or variant thereof of any of the preceding items, a cell manufactured by the method of any of the preceding items, or the cell of any of the preceding items, for the manufacture of a medicament for treating or preventing a disease, disorder, or condition caused by an antigen that is not derived from a subject in the subject.
(40B) The cell of any of the preceding items, wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(41B) The cell of any of the preceding items, wherein the rejection is characterized in that a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a cornea, an eye ball, or a bone marrow.

The present disclosure also provides the following.
(A1) An antibody that inhibits an interaction between CD80 and/or CD86 expressed on a surface of a cell, and CD28 expressed on a surface of another cell, wherein the antibody does not substantially induce the production of a cytokine by immunostimulation.
(A2) The antibody of (A1), wherein the cytokine comprises interferon γ.
(A3) The antibody of (A1) or (A2), wherein an Fc moiety of the antibody is a moiety that does not substantially induce the production of a cytokine by immunostimulation.
(A4) The antibody of any one of (A1) to (A3), wherein a subclass of the antibody is IgG2 or IgG4.
(A5) The antibody of (A4), wherein the subclass of the antibody is IgG4.
A6) The antibody of (A1) or (A2), wherein the antibody lacks an Fc moiety.
(A7) The antibody of (A6), wherein the antibody is an Fab antibody, an F(ab′)₂ antibody, an Fab′ antibody, an Fv antibody, or an scFv antibody.
(A8) The antibody of any one of (A1) to (A7), having an ability to induce immune tolerance.
(A9) The antibody of any one of (A1) to (A8), wherein the cell expressing CD80 and/or CD86 is an antigen presenting cell, and the another cell expressing CD28 is a T cell.
(A10) The antibody of any one of (A1) to (A9), wherein the antibody is an antagonistic anti-CD80 antibody, an antagonistic anti-CD86 antibody, an antagonistic anti-CD28 antibody, or an antagonistic bispecific antibody to CD80 and CD86.
(A11) The antibody of any one of (A1) to (A10), which is a chimeric antibody, a humanized antibody, or a human antibody.
(A12) The antibody of any one of (A1) to (A11), comprising an Fc moiety of IgG2 or IgG4 of a human antibody.
(A13) The antibody of any one of (A1) to (A12), comprising an Fc moiety of IgG4 of a human antibody.
(A14) The antibody of any one of (A1) to (A13), comprising:
   (a) a VH comprising CDRH1 of the amino acid sequence set forth in SEQ ID NO: 25, CDRH2 of the amino acid sequence set forth in SEQ ID NO: 26, and CDRH3 of the amino acid sequence set forth in SEQ ID NO: 27, and a VL comprising CDRL1 of the amino acid sequence set forth in SEQ ID NO: 28, CDRL2 of the amino acid sequence set forth in SEQ ID NO: 29, and CDRL3 set forth in SEQ ID NO: 30; or
   (b) a VH comprising CDRH1 of the amino acid sequence set forth in SEQ ID NO: 31, CDRH2 of the amino acid sequence set forth in SEQ ID NO: 32, and CDRH3 of the amino acid sequence set forth in SEQ ID NO: 33, and a VL comprising CDRL1 of the amino acid sequence set forth in SEQ ID NO: 34, CDRL2 of the amino acid sequence set forth in SEQ ID NO: 35, and CDRL3 set forth in SEQ ID NO: 36.
(A15) The antibody of (A14), comprising:
   (a) a VH having the amino acid sequence set forth in SEQ ID NO: 18 or a variant sequence thereof, and a VL having the amino acid sequence set forth in SEQ ID NO: 20 or a variant sequence thereof; or
   (b) a VH having the amino acid sequence set forth in SEQ ID NO: 22 or a variant sequence thereof, and a VL having the amino acid sequence set forth in SEQ ID NO: 24 or a variant sequence thereof.
(A16) A nucleic acid molecule encoding the amino acid sequence or a portion thereof of the antibody of any one of (A1) to (A15).
(A17) The nucleic acid molecule of (A16) comprising:
   (a) a polynucleotide encoding a VH, having the nucleotide sequence set forth in SEQ ID NO: 17, and a polynucleotide encoding a VL, having the nucleotide sequence set forth in SEQ ID NO: 19; or
   (b) a polynucleotide encoding a VH, having the nucleotide sequence set forth in SEQ ID NO: 21, and a polynucleotide encoding a VL, having the nucleotide sequence set forth in SEQ ID NO: 23.
(A18) A vector having the nucleic acid molecule of (A16) or (A17).
(A19) A cell comprising the nucleic acid molecule of (A16) or (A17) or the vector of (A18).
(A20) A manufacturing method of the antibody of any one of (A1) to (A15), comprising culturing the cell of (A19).
(A21) A cell having immune tolerance induced with the antibody of any one of (A1) to (A15).
(A22) The cell of (A21), wherein the immune tolerance is induced by mixing the antibody, a cell derived from a subject, and an antigen that is not derived from the subject or a material containing the antigen.
(A23) A composition for preparing a cell having immune tolerance induced, comprising at least one of the antibodies of any one of (A1) to (A15).
(A24) The composition of (A23), comprising an antagonistic anti-CD80 antibody, an antagonistic anti-CD86 antibody, an antagonistic anti-CD28 antibody, or an antagonistic bispecific antibody to CD80 and CD86, or any combination thereof.
(A25) A method for manufacturing a cell for treating or preventing a disease, disorder, or condition caused by an antigen that is not derived from a subject, comprising mixing the antibody of any one of (A1) to (A15), a cell derived from the subject, and an antigen that is not derived from the subject or a material containing the antigen.
(A26) The method of (A25), wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(A27) The method of (A25) or (A26), wherein the material containing the antigen is a cell.
(A28) A cell manufactured by the method of any one of (A25) to (A27).
(A29) The cell of (A28), comprising a T cell.
(A30) A cell therapy agent comprising a cell having immune tolerance induced with the antibody of any one of (A1) to (A15), a cell manufactured by the method of (A25) or (A26), or the cell of (A21), (A22), (A28), or (A29).
(A31) A composition for treating or preventing a disease, disorder, or condition caused by an antigen that is not derived from a subject, comprising a cell having immune tolerance induced with the antibody of any one of (A1) to (A15), a cell manufactured by the method of (A25) or (A26), or the cell of (A21), (A22), (A28), or (A29).
(A32) The composition of (A31), wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(A33) The composition of (A32), wherein the rejection is characterized in that a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a corneal, an eye ball, or a bone marrow.
(A34) A method for treating or preventing a disease, disorder, or condition caused by an antigen that is not subject in the subject, comprising derived from a administering to the subject an effective amount of a cell having immune tolerance induced with the antibody of any one of (A1) to (A15), a cell manufactured by the method of (A23) or (A24), or the cell of (A21), (A22), (A28), or (A29).
(A35) The method of (A34), wherein the disease, disorder, or condition is selected from the group consisting of graft rejection, allergy, autoimmune disease, graft-versus-host disease, and immune rejection caused by transplantation of an iPS cell or an ES cell and a cell, tissue, or organ derived from said cells.
(A36) The method of (A35), wherein the rejection is characterized in that a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a cornea, an eye ball, or a bone marrow.

The present disclosure further relates to the following inventions.
(B1) An antibody having a human Fc region, wherein the antibody can inhibit an interaction between CD80 or CD86 expressed on a cell surface and CD28 expressed on another cell surface, and does not promote cytokine production by a human PBMC.
(B2) The antibody of (B1), which can inhibit an interaction between CD80 or CD86 expressed on an antigen presenting cell surface and CD28 expressed on a T cell surface.
(B3) The antibody of (B1) or (B2), which can induce immune tolerance to an antigen by contacting the antibody with a PBMC together with the antigen or a cell having the antigen on a surface.
(B4) The antibody of any one of (B1) to (B3), wherein a cytokine is IFNγ.
(B5) The antibody of any one of (B1) to (B4), which is an anti-CD80 antibody or an anti-CD86 antibody.
(B6) The antibody of any one of (B1) to (B5), which is a chimeric antibody, a humanized antibody, or a human antibody. The antibody of any one of (B1) to (B6), which is a (B7) monoclonal antibody.
(B8) The antibody of any one of (B1) to (B7), whose subclass is IgG2 or IgG4.
(B8A) The antibody of any one of (B1) to (B7), whose subclass is IgG1.
(B8B) The antibody of (B8A), having a heavy chain with the amino acid sequence set forth in a SEQ ID NO. selected from SEQ ID NOs: 38 and 42.
(B8C) The antibody of (B8B), having: (B8C)
   a heavy chain with the amino acid sequence set forth in SEQ ID NO: 38 and a light chain with the amino acid sequence set forth in SEQ ID No: 40, or
   a heavy chain with the amino acid sequence set forth in SEQ ID NO: 42 and a light chain with the amino acid sequence set forth in SEQ ID No: 44.
(B8D) The antibody of any one of (B1) to (B7) and (B8A) to (B8C), having CDRH3 of the amino acid sequence set forth in SEQ ID NO: 55 or 61.
(B8E) The antibody of (B8D), which is
   (Bi) an antibody having CDRH1 of the amino acid sequence set forth in SEQ ID NO: 53, CDRH2 of the amino acid sequence set forth in SEQ ID NO: 54, and CDRH3 of the amino acid sequence set forth in SEQ ID NO: 55, or
   (Bii) an antibody having CDRH1 of the amino acid sequence set forth in SEQ ID NO: 59, CDRH2 of the amino acid sequence set forth in SEQ ID NO: 60, and CDRH3 of the amino acid sequence set forth in SEQ ID NO: 61.
(B8F) The antibody of (Bi) of (B8E), having CDRL1 of the amino acid sequence set forth in SEQ ID NO: 56, CDRL2 of the amino acid sequence set forth in SEQ ID NO: 57, and CDRL3 of the amino acid sequence set forth in SEQ ID NO: 58, or
   the antibody of (Bii) of (B8E), having CDRL1 of the amino acid sequence set forth in SEQ ID NO: 62, CDRL2 of the amino acid sequence set forth in SEQ ID NO: 63, and CDRL3 of the amino acid sequence set forth in SEQ ID NO: 64.
(B8G) The antibody of (B8D), having a heavy chain variable region (VH) with the amino acid sequence set forth in SEQ ID NO: 46 or 50.
(B8H) The antibody of (B8G), having a VH with the amino acid sequence set forth in SEQ ID NO: 46 and a light chain variable region (VL) with the amino acid sequence set forth in SEQ ID NO: 48, or having a VH with the amino acid sequence set forth in SEQ ID NO: 50 and a VL with the amino acid sequence set forth in SEQ ID NO: 52.
(B8I) The antibody of (B8G), having a heavy chain with the amino acid sequence set forth in a SEQ ID NO. selected from SEQ ID NOs: 38 and 42.
(B8J) The antibody of (B8I) having:
   a heavy chain with the amino acid sequence set forth in SEQ ID NO: 38 and a light chain with the amino acid sequence set forth in SEQ ID NO: 40, or
   a heavy chain with the amino acid sequence set forth in SEQ ID NO: 42 and a light chain with the amino acid sequence set forth in SEQ ID NO: 44.
(B9) The antibody of any one of (B1) to (B8), having CDRH3 of the amino acid sequence set forth in SEQ ID NO 27 or 33.
(B10) The antibody of (B9), which is
   (Bi) an antibody having CDRH1 of the amino acid sequence set forth in SEQ ID NO: 25, CDRH2 of the amino acid sequence set forth in SEQ ID NO: 26, and CDRH3 of the amino acid sequence set forth in SEQ ID NO: 27, or
   (Bii) an antibody having CDRH1 of the amino acid sequence set forth in SEQ ID NO: 31, CDRH2 of the amino acid sequence set forth in SEQ ID NO: 32, and CDRH3 of the amino acid sequence set forth in SEQ ID NO: 33.
(B11) The antibody of (Bi) of (B10), having CDRL1 of the amino acid sequence set forth in SEQ ID NO: 28, CDRL2 of the amino acid sequence set forth in SEQ ID NO: 29, and CDRL3 of the amino acid sequence set forth in SEQ ID NO: 30, or
   the antibody of (Bii) of (B10), having CDRL1 of the amino acid sequence set forth in SEQ ID NO: 34, CDRL2 of the amino acid sequence set forth in SEQ ID NO: 35, and CDRL3 of the amino acid sequence set forth in SEQ ID NO: 36.
(B12) The antibody of (B9), having a heavy chain variable region (VH) with the amino acid sequence set forth in SEQ ID NO: 18 or 22.
(B13) The antibody of (B12) having a VH with the amino acid sequence set forth in SEQ ID NO: 18 and a light chain variable region (VL) with the amino acid sequence set forth in SEQ ID NO: 20, or having a VH with the amino acid sequence set forth in SEQ ID NO: 22 and a VL with the amino acid sequence set forth in SEQ ID NO: 24.
(B14) The antibody of (B12), having a heavy chain with the amino acid sequence set forth in a SEQ ID NO. selected from SEQ ID NOs: 2, 6, 10, and 14.
(B15) The antibody of (B14) having:
   a heavy chain with the amino acid sequence set forth in SEQ ID NO: 2 and a light chain with the amino acid sequence set forth in SEQ ID NO: 4;
   a heavy chain with the amino acid sequence set forth in SEQ ID NO: 6 and a light chain with the amino acid sequence set forth in SEQ ID NO: 8;
   a heavy chain with the amino acid sequence set forth in SEQ ID NO: 10 and a light chain with the amino acid sequence set forth in SEQ ID NO: 12; or
   a heavy chain with the amino acid sequence set forth in SEQ ID NO: 14 and a light chain with the amino acid sequence set forth in SEQ ID NO: 16.
(B16) A medical composition comprising the antibody of any one of (B1) to (B15).
(B17) The medical composition of (B16), comprising an anti-CD80 antibody anti-CD86 antibody.
(B18) The medical composition of (B16) or (B17) used for inducing immune tolerance.
(B19) The medical composition of (B18) used for inducing immune tolerance in a cell collected from a patient receiving organ transplantation.
(B20) The medical composition of (B19) or (B20), wherein an organ to be transplanted is a heart, a kidney, or a liver.
(B21) The medical composition of (B18) used for inducing immune tolerance in a patient with an autoimmune disease or an allergic disease.
(B22) A nucleic acid molecule encoding the amino acid sequence of the antibody of any one of (B12) to (B15).
(B23) The nucleic acid molecule of (B22) comprising a polynucleotide encoding a VH with the nucleotide sequence set forth in any one selected from SEQ ID NOs: 1, 5, 9, and 13 and a polynucleotide encoding a VL with the nucleotide sequence set forth in any one selected from SEQ ID NOs: 3, 7, 11, and 15.
(B24) A vector having the nucleic acid molecule of (B22) or (B23).
(B25) A host cell having the vector of (B24).
(B26) A method of manufacturing the antibody of any one of (B1) to (B15), comprising culturing the host cell of (B25).
(B27) A method of inducing immune tolerance to an antigen of a PBMC, comprising contacting the antibody of any one of (B1) to (B15), an antigen for which immune tolerance is to be induced or a cell with the antigen on a surface, and the PBMC.
(B28) The inducing method of (B27), which is performed ex (B28) vivo.
(B29) The method of (B27) or (B28), wherein the PBMC is a cell collected from a patient receiving organ transplantation.
(B30) The method of (B27) or (B28), wherein the PBMC is a cell collected from a patient with an autoimmune disease or an allergic disease.
(B31) A PBMC having immune tolerance induced by the method of any one of (B27) to (B30).
(B32) A cell therapy agent comprising the PBMC of (B30) as an active ingredient.
(B33) The cell therapy agent of (B32) for suppressing rejection in organ transplantation.
(B34) The cell therapy agent of (B33), wherein a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a cornea, an eye ball, or a bone marrow.
(B35) The cell therapy agent of (B32) for treating an autoimmune disease or an allergic disease.
(B36) A method of suppressing a rejection in organ transplantation, comprising administering the antibody of any one of (B1) to (B15) to a patient receiving organ transplantation.
(B37) The method of (B36), wherein a transplanted organ is a kidney, a liver, a heart, skin, a lung, a pancreas, an esophagus, a stomach, a small intestine, a large intestine, a nerve, blood, a blood cell including an immune system cell, a bone, a cartilage, a blood vessel, a cornea, an eye ball, or a bone marrow.
(B37) A method of treating an autoimmune disease or an allergic disease, comprising administering the antibody of any one of (B1) to (B15) to a patient with an autoimmune disease or an allergic disease.
(B38) A method of suppressing rejection in organ transplantation, comprising administering the cell therapy agent of any one of (B32) to (B34) to a patient receiving organ transplantation.
(B39) A method of treating an autoimmune disease or an allergic disease, comprising administering the cell therapy agent of (B32) or (B35) to a patient with an autoimmune disease or an allergic disease.
(B40) The method of any one of (B27) to (B30), further comprising administering a with immune tolerance induced to a patient.
(B41) The method of (B40), comprising administering to a patient receiving organ transplantation a PBMC derived from a patient with immune tolerance to an organ to be transplanted induced.
(B42) The method of (B40), comprising administering to a patient with an autoimmune disease or an allergic disease a PBMC derived from a patient with immune tolerance to an antigen, which is a cause of an autoimmune disease or an allergic disease induced.

The present disclosure is intended so that one or more of the features described above can be provided not only as the explicitly disclosed combinations, but also as other combinations thereof. Additional embodiments and advantages of the present disclosure are recognized by those skilled in the art by reading and understanding the following detailed description as needed.

### Advantageous Effects of Invention

The antibody of the present disclosure suppresses IFNγ production while maintaining the ability to induce immune tolerance. In particular, the antibodies of subclass IgG2 or IgG4 of the present disclosure can manufacture a cell with induced immune tolerance without producing IFNγ.

### BRIEF DESCRIPTION OF DRAWINGS

FIG. 1 shows results of a mixed lymphocyte reaction (³H-thymidine incorporation) in the presence of anti-human CD80 and anti-CD86 antibodies (IgG1). Naïve; no donor added, no antibody added, Allo; donor added, no antibody added. 10, 1, and 0.1 are concentrations (μg/ml) of each added antibody. The vertical axis indicates ³H-thymidine incorporation, based on no antibody added (Allo).

FIG. 2 shows results of a mixed lymphocyte reaction (IFNγ production) in the presence of anti-human CD80 and anti-CD86 antibodies (IgG1). The vertical axis indicates the concentration in a medium (pg/ml).

FIG. 3 shows results of a mixed lymphocyte reaction (³H-thymidine incorporation) in the presence of anti-human CD80 and anti-CD86 antibodies (IgG1). The vertical axis indicates ³H-thymidine incorporation, based on no antibody added (Allo).

FIG. 4 shows results of a mixed lymphocyte reaction (IFNγ production) in the presence of anti-human CD80 and anti-CD86 antibodies (IgG1). Top row: with donor stimulation, bottom row: no donor stimulation. * represents 50000 pg/mL or greater. The vertical axis indicates the concentration in a medium (pg/ml).

FIG. 5 shows results of a mixed lymphocyte reaction (³H-thymidine incorporation) in the presence of anti-CD80 and anti-CD86 (IgG2, antibodies IgG4). The vertical axis indicates ³H-thymidine incorporation, based on no antibody added (Allo). 10, 3, and 1 are concentrations of each added antibody (μg/ml).

FIG. 6 shows results of a mixed lymphocyte reaction (IFNγ production) in the presence of anti-CD80 and anti-CD86 antibodies (IgG1, IgG2, IgG4). The vertical axis indicates the concentration in a medium (pg/ml).

FIG. 7 shows results of a mixed lymphocyte reaction (³H-thymidine incorporation) upon addition of a cell obtained in the presence of anti-CD80 and anti-CD86 antibodies (IgG1, IgG2, IgG4). The vertical axis indicates ³H-thymidine incorporation, based on no antibody added (Allo). ½, ¼, and ⅛ are ratios of mixture of cells obtained in the presence of anti-CD80 and anti-CD86 antibodies (IgG1, IgG2, IgG4) to naïve responder cells.

FIG. 8 shows results of a mixed lymphocyte reaction (IFNγ production) in the presence of chimeric anti-CD80 antibodies and chimeric anti-CD86 antibodies. The vertical axis indicates the concentration in a medium (pg/ml).

FIG. 9 shows results of a mixed lymphocyte reaction (³H-thymidine incorporation) upon addition of a cell obtained in the presence of chimeric anti-CD80 and chimeric anti-CD86 antibodies. The vertical axis indicates ³H-thymidine incorporation, based on no antibody added (Allo). ½, ¼, ⅛, and 1/16are ratios of mixture of cells obtained in the presence of chimeric anti-CD80 and chimeric anti-CD86 antibodies to naïve responder cells.

### DESCRIPTION OF EMBODIMENTS

The terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present invention pertains. In case of a contradiction, the present specification (including the definitions) takes precedence.

### Definitions of Terms

As used herein, “about” refers to a range of ±10% of the subsequent numerical value.

As used herein, “immune tolerance” refers to a state where a specific immune response to a specific antigen is not exhibited or a specific immune response is suppressed. Immune tolerance can also refer to either or both of a state where an immune cell (especially T cell) does not exhibit a specific immune response to a specific antigen or a specific immune response is suppressed, and a state where a human does not exhibit a specific immune response to a specific antigen or a specific immune response is suppressed. Immune tolerance has drawn attention because elicitation of immune tolerance makes it possible to treat immune rejection or treat allergies. As used herein, “anergy” refers to a state where costimulation is not inputted when an antigen is presented from an antigen presenting cell so that a cell cannot respond upon stimulated under the condition with costimulation the next time. As used herein, “PBMC (or T cell) with immune tolerance induced” is synonymous with “anergic PBMC (or T cell)”.

As used herein, “subject” includes domesticated animals (e.g., cows, sheep, cats, dogs, and horses), primates (e.g., humans and non-human primates such as monkeys), rabbits, and rodents (e.g., mice and rats). In a specific embodiment, the subject is a human.

As used herein, “immunostimulation” refers to stimulation applied to cells in the immune system (e.g., macrophage, neutrophil, natural killer (NK) cell, T cell, B cell, etc.) or proliferation of cells in the immune system. Cytokine (e.g., INF) production or cytotoxic activity is induced by stimulation of cells in the immune system.

As used herein, “do not substantially induce the production of a cytokine by immunostimulation” refers to inducing cytokine production of about 20% or less of the amount of cytokines produced by cells (e.g., macrophage, neutrophil, natural killer (NK) cell, T cell, B cell, etc.) derived from a subject in the presence of an antigen and in the absence of the antibody of the present disclosure.

As used herein, “antibody” broadly refers to a molecule or a group thereof that can specifically bind to a specific epitope on an antigen. As used herein, “antibody” can be broadly a full-length antibody (i.e., antibody with an Fc moiety) or an antibody lacking an Fc moiety. An antibody lacking an Fc moiety only needs to be able to bind to an antigen of interest. Examples of such an antibody include, but are not limited to, Fab antibodies, F(ab′)₂ antibodies, Fab′ antibodies, Fv antibodies, and scFv antibodies, and the like. Antibodies also include modified and unmodified antibodies. For modified antibodies, an antibody can be bound to various molecules such as polyethylene glycol. A modified antibody can be obtained by applying a chemical modification to an antibody using a known method.

As used herein, “antibody” narrowly refers to immunoglobulin or a group thereof that can specifically bind to a specific epitope on an antigen. A variant form thereof is referred to as a “variant of an antibody”. As used herein, “antibody” can be narrowly a full-length antibody (i.e., antibody with an Fc moiety). A “variant of an antibody” herein can be a variant lacking an Fc moiety of an antibody described above. Therefore, as used herein, an antibody can also be narrowly referred to as a full-length antibody, and a variant of an antibody can also be referred to as a variant of a full-length antibody. A variant lacking an Fc moiety only needs to be able to bind to an antigen of interest. Examples of such a variant include, but are not limited to, Fab antibodies, F(ab′)₂ antibodies, Fab′ antibodies, Fv antibodies, and scFv antibodies, and the like. Variants of an antibody also include modified antibodies and unmodified antibodies. For modified antibodies, an antibody can be bound to various molecules such as polyethylene glycol. A modified antibody can be obtained by applying a chemical modification to an antibody using a known method.

In one embodiment of the present disclosure, “polyclonal antibody” can be generated, for example, by administering an immunogen comprising an antigen of interest to a mammal (e.g., rat, mouse, rabbit, cow, monkey, or the like), avian, or the like to induce the production of a polyclonal antibody specific to an antigen. An immunogen can be administered through one or more immunologic agents, and infusion of an adjuvant when desired. An adjuvant can be used to increase immune responses and can include Freund's adjuvant (complete or incomplete), mineral gel (aluminum hydroxide or the like), surfactant (lysolecithin or the like), or the like. The immunization protocol is known in the art and can be performed by any method that induces an immune response in accordance with the selected host organism (“Tanpakushitsu Jikken Handobukku [Protein experiment handbook], Yodosha (2003): 86-91”).

In one embodiment of the present disclosure, “monoclonal antibody” encompasses individual antibodies constituting a population that are identical antibodies corresponding to substantially a single epitope, except for antibodies having a mutation that can occur naturally in small amounts. Further, individual antibodies constituting a population may be antibodies that are substantially the same except for antibodies having a mutation that can occur naturally in small amounts. Monoclonal antibodies are highly specific, which are different from common polyclonal antibodies that typically include different antibodies corresponding to different epitopes and/or different antibodies corresponding to the same epitope. In addition to their specificity, monoclonal antibodies are useful in that they can be synthesized from a hybridoma culture which is not contaminated with other immunoglobulins. The description “monoclonal” may indicate a characteristic of being obtained from a substantially homogeneous antibody population. However, such a description does not mean that antibodies must be produced by a specific method. For example, monoclonal antibodies may be prepared by a method similar to the hybridoma method described in “Kohler G, Milstein C., Nature. 1975 Aug. 7; 256 (5517): 495-497”. Alternatively, monoclonal antibodies may be prepared by a method similar to the recombinant method described in U.S. Pat. No. 4,816,567. Monoclonal antibodies may also be isolated from a phage antibody library using a method similar to the technology that is described in “Clackson et al., Nature. 1991 Aug. 15; 352 (6336): 624-628.” or “Marks et al., J Mol Biol. 1991 Dec. 5; 222(3): 581-597”. Monoclonal antibodies may also be prepared by the method described in “Tanpakushitsu Jikken Handobukku [Protein experiment handbook], Yodosha (2003): 92-96”.

In one embodiment of the present disclosure, “chimeric antibody” is, for example, a variable region of an antibody linked to a constant region of an antibody between xenogenic organisms and can be constructed by a genetic engineering technology. A mouse-human chimeric antibody can be prepared by, for example, the method described in “Roguska et al., Proc Natl Acad Sci USA. 1994 Feb. 1; 91(3): 969-973.” For example, the basic method of preparing a mouse-human chimeric antibody links a mouse leader sequence and a variable region sequence in a cloned cDNA with a sequence encoding a human antibody constant region already present in an expression vector of a mammalian cell. After linking the mouse leader sequence and variable region sequence in a cloned cDNA with the sequence encoding a human antibody constant region, the resultant sequence may be linked to a mammalian cell expression vector. A fragment of a human antibody constant region can be from any human antibody H chain constant region and human antibody L chain constant region. Examples of human H chain fragment include Cγ1, Cγ2, Cγ3, or Cγ4, and examples of L chain fragment include Cλ or Cκ.

In one embodiment of the invention, “humanized antibody” is, for example, an antibody, which has one or more CDRs derived from a nonhuman species, a framework region (FR) derived from a human immunoglobulin, and a constant region derived from human immunoglobulin and binds to a desired antigen. Antibodies be can humanized using various approaches known in the art (Almagro et al., Front Biosci. 2008 Jan. 1; 13:1619-1633). Examples thereof include CDR grafting (Ozaki et al., Blood. 1999 Jun. 1; 93(11): 3922-3930.), Re-surfacing (Roguska et al., Proc Natl Acad Sci U S A. 1994 Feb. 1; 91(3): 969-973.), FR shuffle (Damschroder et al., Mol Immunol. 2007 April; 44(11): 3049-3060. Epub 2007 Jan. 22.) and the like. An amino acid residue of a human FR region may be substituted with a corresponding residue from a CDR donor antibody in order to alter (preferably in order to improve) the antigen bond. The FR substitution can be performed by a method that is well known in the art (Riechmann et al., Nature. 1988 Mar. 24; 332 (6162): 323-327.) For example, an FR residue important for antigen binding may be identified by modeling an interaction between a CDR and an FR residue. Further, an abnormal FR residue at a specific position may be identified by sequence comparison.

In one embodiment of the invention, “human antibody” is, for example, an antibody in which a region comprising a variable region and constant region of a heavy chain and variable region and constant region of a light chain constituting the antibody is derived from a gene encoding a human immunoglobulin. Examples of main preparation methods include a method using a transgenic mouse for preparing human antibodies, phage display method, and the like. A method using a transgenic mouse for preparing human antibodies produces human antibodies with diverse antigen binding capabilities instead of mouse antibodies if a functional human Ig gene is introduced into an endogenous Ig knockout mouse. Furthermore, this mouse can be immunized to obtain human monoclonal antibodies by a conventional hybridoma method. This can be prepared, for example, by the method described in “Lonberg et al., Int Rev Immunol. 1995; 13(1): 65-93.” The phage display method is a system that typically expresses an exogenous gene as a fusion protein such that phage infectivity is not lost on the N-terminus side of a coat protein (g3p, g10p, or the like) of fibrous phage such as an E. coli virus M13 or T7. Antibodies can be prepared, for example, by the method described in “Vaughan et al., Nat Biotechnol. 1996 March; 14(3): 309-314”.

As used herein, “variant sequence” refers to a sequence with at least one amino acid residue alteration (substitution, addition, or deletion) to the original sequence for an amino acid sequence, and refers to an alteration (substitution, addition, or deletion) of at least one base that would not result in a frame shift for a nucleic acid sequence. A variant sequence has a function similar to the original sequence, which is for example at least 80%, at least 85%, at least 90%, at least 95%, at least 96%, at least 97%, at least 98%, or at least 99% identical to the original sequence. If the original sequence is an antibody, a variant sequence preferably does not have an alternation within a CDR, but can have one or more alternations, preferably 3 or less alternations, more preferably 2 or less alterations, and most preferably one alternation, as long as the binding ability or function of the original antibody is maintained. When an alternation is introduced within a CDR, those skilled in the art can select an appropriate alternation so that the binding ability or function of the original antibody is maintained.

As used herein, “cell derived from a subject” refers to a cell obtained from a subject administered with the composition of the present disclosure or a cell derived from a cell obtained from the subject. As used herein, “antigen derived from a subject” refers to an antigen produced by a subject themselves which induces an immune response, such as an antigen produced by a subject themselves which causes an autoimmune disease in a subject with the autoimmune disease. As used herein, “antigen that is not derived from a subject” refers to an exogenous antigen that can induce an immune response. As used herein, “antigen-containing material that is not derived from a subject” refers to any substance or collection of substances comprising an antigen that is not derived from a subject. Examples thereof include a cell, cell population, tissue, and the like expressing an antigen that is not derived from a subject.

As used herein, “graft rejection” refers to the immune system of a subject attacking, damaging, or destroying a transplanted organ, tissue, or cell in a subject receiving transplantation of the organ, tissue, or cell.

As used herein, “allergy” refers to a hyperactive immune response to an antigen that is not derived from a subject. An antigen that is not derived from a subject, which induces an allergy, is also referred to as an allergen. Examples thereof include, but are not limited to, tick antigen, egg white antigen, milk antigen, wheat antigen, peanut antigen, soybean antigen, buckwheat antigen, sesame antigen, rice antigen, crustacean antigen, kiwi antigen, apple antigen, banana antigen, peach antigen, tomato antigen, tuna antigen, salmon antigen, mackerel antigen, beef antigen, chicken meat antigen, pork antigen, feline dander antigen, insect antigen, pollen antigen, dog dander antigen, fungal antigen, bacterial antigen, latex, hapten, metal, and the like.

As used herein, “autoimmune disease” refers to any disease in which the immune system exerts an undesirable immune response on its own cell, tissue, or organ. Examples of an autoimmune disease include, but are not limited to, rheumatoid arthritis, multiple sclerosis, type 1 diabetes, inflammatory bowel disease (e.g. Crohn's disease or ulcerative colitis), systemic lupus erythematosus, psoriasis, scleroderma, autoimmune thyroid disease, alopecia areata, Graves' disease, Guillain Barre syndrome, celiac disease, Sjogren's syndrome, rheumatic fever, gastritis, autoimmune atrophic gastritis, autoimmune hepatitis, pancreatitis, ovitis, orchitis, uveitis, lens-induced uveitis, myasthenia gravis, primary myxedema, pernicious anemia, autoimmune hemolytic anemia, Addison's disease, scleroderma, Goodpasture syndrome, nephritis (e.g., glomerulonephritis), psoriasis, pemphigus vulgaris, pemphigoid, sympathetic ophthalmia, idiopathic thrombocytopenia purpura, idiopathic leukopenia, Wegener's granulomas, and polymyositis/dermatomyositis.

As used herein, “graft-versus-host” refers to a transplanted organ, tissue, or cell attacking, damaging, or destroying a cell, tissue, or organ of a subject who received transplantation due to an immune response.

As used herein, “immune rejection caused by transplantation of an iPS cell or ES cell, or a cell, tissue, or organ derived from said cells” refers to an immune rejection resulting from an antigen of an iPS cell or ES cell, or an antigen of a cell, tissue, or organ derived from an iPS cell or ES cell.

### Preferred Embodiments

The preferred embodiments are described hereinafter. It is understood that the embodiments are exemplification of the present disclosure, so that the scope of the present disclosure is not limited to such preferred embodiments. It is understood that those skilled in the art can refer to the following preferred embodiments to readily make modifications or changes within the scope of the present disclosure. Any of these embodiments can be appropriately combined by those skilled in the art.

As a result of attempting to induce non-immune response (anergy) using an antibody having a human Fc region that inhibits the interaction between CD80 and/or CD86 and CD28, the inventors found that this activates a cell involved in the immune system such as the macrophage, neutrophil, or natural killer (NK) cell and induces the release of a humoral factor such as interleukin or interferon (IFN) responsible for immune responses. The inventors newly discovered that induction of non-immune response (anergy) using an antibody having a human Fc region has a problem of attenuating the effect of immune tolerance because release of these humoral factors induces unpreferable activation of the immune system, which is the opposite of immune tolerance, and found that the effect of immune tolerance can be improved by adjusting the human Fc region.

Specifically, a chimeric antibody that inhibits the interaction between CD80 and/or CD86 and CD28 and induces immune tolerance was prepared by immunizing Balb/c mice with a recombinant/human CD80/CD86-Fc fusion protein, then extracting mRNA from the spleen of the mice, synthesizing a CDNA, selecting a Fab antibody with high affinity to CD80/CD86 identified with a phase display library that was constructed using a gene amplified by RT-PCR, and then imparting a human Fc region by genetic engineering.

To minimize rejection to an antibody used for induction of immune tolerance, the inventors humanized a chimeric antibody with the CDR grafting method developed by Xoma to prepare a humanized antibody (subclass; IgG1). The resulting humanized anti-CD80 antibody and anti-CD86 antibody (subclass; IgG1) were added to a mixed lymphocyte reaction with PBMCs (peripheral blood mononuclear cells) harvested from two volunteers assuming a donor or recipient to evaluate the suppression function upon the same antigen stimulation of the induced cells. Specifically, PBMCs were harvested from one volunteer, irradiated with 30 Gy radiation, and co-cultured for 7 days with PBMCs harvested from another volunteer in the presence of a humanized anti-CD80/CD86 antibody to induce a donor antigen specific anergic cell. The induced cells were added at a certain ratio to mixed lymphocytes from the same recipient and donor, and the ability to suppress division and proliferations of cells with antigen stimulation was measured as the amount of tritium labeled thymidine incorporation. It was confirmed, as a result, that humanized anti-CD80 antibodies and anti-CD86 antibodies significantly suppress the amount of thymidine incorporation, and cells induced by such antibodies have newly acquired a suppression function.

However, when the amount of IFNγ in the supernatant after 7 days of culture was measured, IFNγ was produced upon addition of an antibody, even without a non-antigen stimulation. It was also revealed that IFNγ generally produced upon allo antigen simulation due to addition of PBMCs irradiated with radiation is in the supernatant at almost the same amount after addition of an antibody.

In this regard, the inventors found that a cell inducing immune tolerance, without producing IFNγ, can be obtained by using humanized anti-CD80/CD86 antibodies of subclass IgG2 or IgG4 as a result of diligent study to obtain an antibody that does not induce IFNγ production while maintaining the ability to induce immune tolerance.

### Antibodies

In one embodiment of the present disclosure, the present disclosure provides an antibody that inhibits an interaction between CD80 and/or CD86 expressed on a surface of a cell, and CD28 expressed on a surface of another cell, wherein the antibody does not substantially induce the production of a cytokine by immunostimulation. The antibody of the present disclosure can be an anti-CD80 antibody and/or anti-CD86 antibody, a bispecific antibody to CD80 and CD86, an anti-CD28 antibody, or a mixture thereof. In some embodiments, an Fc moiety of the antibody can be a moiety that does not substantially induce the production of a cytokine by immunostimulation. In a specific embodiment, a subclass of the antibody of the present disclosure is IgG2 or IgG4. The antibody of the present disclosure whose subclass is IgG2 or IgG4 surprisingly did not induce any production of cytokine due to an immune system cell. Since the antibody used in the present disclosure can result in an undesirable effect by activating a receptor, an antagonistic antibody is preferable.

In some embodiments, an antibody that does not substantially induce the production of a cytokine by immunostimulation can induce cytokine production of about 20% or less, preferably about 10% or less, more preferably about 5% or less, and most preferably about 0% of the amount of cytokines produced by a cell derived from a subject (e.g., macrophage, neutrophil, natural killer (NK) cell, T cell, B cell, or the like) in the absence of the antibody of the present disclosure.

As used herein, “antagonistic (antibody)” is synonymous with an antagonist (antibody), inhibition (antibody), blocking (antibody), or the like, which (is an antibody for antibodies) has the function to inhibit the targeted function or signaling function to attenuate or inactivate the action of a living organism.

As induction of the production of cytokines was unexpectedly suppressed by changing the subclass of an antibody from IgG1 to IgG2 or IgG4 as described above. Although not wishing to be bound by any theory, it is understood that induction of cytokine production is due to an Fc moiety of an IgG1 antibody because the function mediated through the Fc moiety varies among IgG subclasses. Thus, in another embodiment, the antibody of the present disclosure can be an antibody lacking an Fc moiety. Examples of such an antibody include, but are not limited to, an Fab antibody, an F(ab′)₂ antibody, an Fab′ antibody, an Fv antibody, an scFv antibody, and the like.

In some embodiments, the cell expressing CD80 and/or CD86 can be an antigen presenting cell, and the another cell expressing CD28 can be a T cell.

In another aspect of the present disclosure, the antibody of the present disclosure can inhibit an interaction between CD80 and/or CD86 expressed on a cell surface, and CD28 expressed on another cell surface. Whether an interaction between CD80 and/or CD86 expressed on a cell surface and CD28 expressed on another cell surface can be inhibited can be determined by, for example, coimmunoprecipitation. Specifically, these proteins are bound and then immunoprecipitated using an antibody that binds to one of the proteins (bait), and the other immunoprecipitated protein (prey) is measured. If the prey contained in immunoprecipitation is decreased when a candidate antibody is added compared to a control without a candidate antibody, the candidate antibody is determined as inhibiting the binding between the proteins. A prey contained in an immunoprecipitation can be measured by labeling the prey or the like as needed. The amount of prey bound to a carrier can be compared by binding one of the protein (bait) to the carrier using pull-down assay method and contacting the other protein (prey) in the presence/absence of a candidate antibody.

Preferably, the antibody of the present disclosure can inhibit an interaction between CD80 and/or CD86 expressed on an antigen presenting cell surface and CD28 expressed on a T cell surface. Whether a candidate antibody inhibits an interaction between CD80 and/or CD86 expressed on an antigen presenting cell surface and CD28 expressed on a T cell surface can be determined by contacting the antigen presenting cell expressing CD80 and/or CD86 with a T cell expressing CD28 in the presence of a candidate antibody and determining that an interaction between CD80 and/or CD86 expressed on the antigen presenting cell surface and CD28 expressed on the T cell surface is inhibited if the T cell is not activated.

Further, the antibody of the present disclosure is characterized by not promoting cytokine production by human PBMCs. Whether a candidate antibody does not promote cytokine production by a human PBMC can be determined by contacting the candidate antibody with the human PBMC in a medium and measuring the amount of cytokines released in the medium. A candidate antibody with lower/greater amount of cytokines released in the presence of the candidate antibody compared to the amount of cytokines released by a human PBMC in the absence of antibody an is determined as not promoting/promoting cytokine production by a human PBMC. Examples of cytokines include interleukins such as IL-1β, IL-2, IL-4, IL-5, IL-6, IL-8, IL-9, IL-14, IL-15, IL-17, and IL-18; chemokines such as CC, CXC, CX3C, and C; interferons such as IFNα, IFNβ, and IFNγ; and cytotoxic agents such as TNFα. Cytokines are preferably inflammatory cytokines and more preferably IFNγ. Cytokines can be measured using a commercially available kit. For example, IFNγ can be measured using Biolegend's Human IFNγ ELISA MAX™ Deluxe.

Preferably, the antibody of the present disclosure can be contacted with a PBMC together with an antigen or a cell having an antigen on a surface to induce immune tolerance to the antigen. Whether a candidate antibody can induce immune tolerance to an antigen by contacting the antibody with a PBMC together with the antigen or a cell having the antigen on a surface can be determined by contacting the antigen or cell having the antigen on a surface with a PBMC for several days to 7 days in the presence of the candidate antibody, then adding ³H-thymidine, and removing the ³H-thymidine in culture 16 to 20 hours after adding the ³H-thymidine, and then measuring the amount of ³H-thymidine incorporation by the PBMC. If the amount of ³H-thymidine incorporation by a PBMC is low when the candidate antibody is added compared to a control without deployment of the candidate antibody, the antibody can be determined as capable of inducing immune tolerance to the antibody by contacting the antibody with the PBMC together with an antigen or a cell having an antigen on a surface.

As used herein, “immune tolerance” is a condition where a specific immune response to a specific antigen is not exhibited or a specific immune response is suppressed. Immune tolerance can mean either or both a state where an immune cell (especially a T cell) does not exhibit a specific immune response to a specific antigen or a specific immune response is suppressed, and a state where a human does not exhibit a specific immune response to a specific antigen or a specific immune response is suppressed. As used herein, “anergy” refers to a T cell that can no longer respond even when stimulated under the condition with costimulation the next time due to no input of costimulation when an antigen is presented from an antigen presenting cell. As used herein, “PBMC (or T cell) with immune tolerance induced” and “anergic PBMC (or T cell)” are synonymous.

Preferably, the present disclosure relates to an anti-CD80 antibody and anti-CD86 antibody. The anti-CD80 antibody and anti-CD86 antibody herein specifically binds to CD80 and Cd86, respectively. As used herein, an antibody's “specific” binding refers to the antibody binding at a substantially higher affinity to a protein of interest (CD80 or CD86) than the affinity to other proteins or peptides. In this regard, “binding at a substantially higher affinity” refers to higher affinity to the extent that a specific protein or peptide of interest can be distinctly detected from other proteins or peptides by a desirable measuring apparatus or method. For example, substantially higher affinity can mean about 3-fold or greater, about 4-fold or greater, about 5-fold or greater, about 6-fold or greater, about 7-fold or greater, about 8-fold or greater, about 9-fold or greater, about 10-fold or greater, about 20-fold or greater, about 30-fold or greater, about 40-fold or greater, about 50-fold or greater, or about 100-fold or greater as the intensity (e.g., fluorescence intensity) detected by ELISA or EIA.

Examples of association rate constant (Ka1) in the binding of the antibody of the present disclosure with CD80, CD86, or CD28 include about 1×10⁴ Ms⁻¹ or greater, about 1×10⁵ Ms⁻¹ or greater, and about 5×10⁵ Ms⁻¹ or greater. Examples of dissociation rate constant (Kd1) in the binding of the antibody of the present disclosure with CD80, CD86, or CD28 include about 1×10⁻³ Ms⁻¹ or less and about 1×10⁻⁴ Ms⁻¹ or less. Examples of association constant (KD) in the binding of the antibody of the present disclosure with CD80, CD86, or CD28 include about 1×10⁻⁸ (M) or less, about 5×10⁻⁸ (M) or less, about 5×10⁻⁹ (M) or less, and about 5×10⁻⁹ (M) or less. For the association rate constant (Ka1), dissociation rate constant (Kd1), and association constant (KD) of an antibody herein, BIACORE (GE Healthcare Bio-Sciences KK, BIACORE-X100) can be used according to the manual the provided by manufacturer to immobilize biotinylated CD80, CD86, or CD28 or a cell expressing the same on an SA chip then allowing an antibody to be tested to flow to measure the association rate constant Ka1 and dissociation rate constant Kd1, and using bivalent fitting to measure the association constant KD.

The antibody of the present disclosure can be a polyclonal antibody or a monoclonal antibody, but is preferably a monoclonal antibody. In the present disclosure, “monoclonal antibodies” are antibodies with a uniform structure, reacting to a single antigenic determinant. Furthermore, the antibody of the present disclosure encompasses an antibody having an amino acid sequence of an antibody of a non-human animal and an amino acid sequence of an antibody derived from a human and a human antibody. Examples of antibodies of a non-human animal include antibodies of a mouse, rat, hamster, guinea pig, rabbit, dog, monkey, sheep, goat, camel, chicken, duck, or the like, preferably antibodies of an animal with which a hybridoma can be prepared, and more preferably antibodies of a mouse, rat, or rabbit. Examples of antibodies with an amino acid sequence of an antibody of a non-human animal and an amino acid sequence of an antibody derived from a human include human type chimeric antibodies and humanized antibodies. The “chimeric antibody” described above is a non-human animal derived antibody altered by genetic engineering so that a constant region of an antibody specifically binding to an antigen of interest (CD80, CD86, or CD28) is the same constant region as a human antibody. A chimeric antibody is preferably a human mouse chimeric antibody (see EP Patent Application Publication No. 0125023). A “humanized antibody” is a non-human animal derived antibody altered by genetic engineering so that the primary structure other than the H-chain and L-chain complementarity-determining regions (CDR) of an antibody specifically biding to CD80, CD86, or CD28 is a primary structure corresponding to a human antibody. In this regard, CDR can be defined by one of Kabat et al. (“Sequences of Proteins of Immunological Interest”, Kabat, E. et al., U.S. Department of Health and Human Services, 1983), or Chothia et al. (Chothia & Lesk (1987) J. Mol. Biol., 196: 901-917). A “human antibody” is a human antibody, which is a fully-human derived antibody gene expression production. Examples thereof include monoclonal antibodies prepared using a transgenic animal introduced with a gene involved with antibody production of a human (EP Patent Application Publication No. 0546073) and the like. When, for example, the antibody of the present disclosure is used in treatment, prevention, or diagnosis using the antibody by administration into the body, the antibody of the present disclosure is preferably a chimeric antibody of human/non-human animal, humanized antibody, or human antibody.

Preferably, the immunoglobulin class of the antibody of the present disclosure is IgG. The subclass of the antibody of the present disclosure is preferably IgG2 or IgG4. More preferably, the subclass of the antibody of the present disclosure is IgG4. Alternatively, the subclass of the antibody of the present disclosure is preferably IgG2. The antibody of the present disclosure can be a monospecific, bispecific (bispecific antibody), trispecific (trispecific antibody) (e.g., WO 1991/003493), tetraspecific (tetraspecific antibody), or greater multi-specific (multi-antigen specific).

In one embodiment, the antibody of the present disclosure has CDRH3 of the amino acid sequence set forth in SEQ ID NO: 27 or SEQ ID NO: 33. Preferably, the antibody of the present disclosure has CDRH1 of the amino acid sequence set forth in SEQ ID NO: 25, CDRH2 of the amino acid sequence set forth in SEQ ID NO: 26, and CDRH3 of the amino acid sequence set forth in SEQ ID NO: 27, or CDRH1 of the amino acid sequence set forth in SEQ ID NO: 31, CDRH2 of the amino acid sequence set forth in SEQ ID NO: 32, and CDRH3 of the amino acid sequence set forth in SEQ ID NO: 33. More preferably, the antibody of the present disclosure has CDRH1 of the amino acid sequence set forth in SEQ ID NO: 25, CDRH2 of the amino acid sequence set forth in SEQ ID NO: 26, and CDRH3 of the amino acid sequence set forth in SEQ ID NO: 27, and CDRL1 of the amino acid sequence set forth in SEQ ID NO: 28, CDRL2 of the amino acid sequence set forth in SEQ ID NO: 29, and CDRL3 of the amino acid sequence set forth in SEQ ID NO: 30, or CDRH1 of the amino acid sequence set forth in SEQ ID NO: 31, CDRH2 of the amino acid sequence set forth in SEQ ID NO: 32, and CDRH3 of the amino acid sequence set forth in SEQ ID NO: 33 and CDRL1 of the amino acid sequence set forth in SEQ ID NO: 34, CDRL2 of the amino acid sequence set forth in SEQ ID NO: 35, and CDRL3 of the amino acid sequence set forth in SEQ ID NO: 36.

In one embodiment, the antibody of the present disclosure can be an antibody, or a variant thereof, having a heavy chain with the amino acid sequence set forth in SEQ ID NO: 38 or 42 or a variant sequence thereof, and a light chain with the amino acid sequence set forth in SEQ ID NO: 40 or 44 or a variant sequence thereof. Preferably, the antibody of the present disclosure can be an antibody, or a variant thereof, comprising (a) a VH comprising CDRH1 of the amino acid sequence set forth in SEQ ID NO: 53 or a variant sequence thereof, CDRH2 of the amino acid sequence set forth in SEQ ID NO: 54 or a variant sequence thereof, and CDRH3 of the amino acid sequence set forth in SEQ ID NO: 55 or a variant sequence thereof, and a VL comprising CDRL1 of the amino acid sequence set forth in SEQ ID NO: 56 or a variant sequence thereof, CDRL2 of the amino acid sequence set forth in SEQ ID NO: 57 or a variant sequence thereof, and CDRL3 of the amino acid sequence set forth in SEQ ID NO: 58 or a variant sequence thereof, or (b) a VH comprising CDRH1 of the amino acid sequence set forth in SEQ ID NO: 59 or a variant sequence thereof, CDRH2 of the amino acid sequence set forth in SEQ ID NO: 60 or a variant sequence thereof, and CDRH3 of the amino acid sequence set forth in SEQ ID NO: 61 or a variant sequence thereof, and a VL comprising CDRL1 of the amino acid sequence set forth in SEQ ID NO: 62 or a variant sequence thereof, CDRL2 of the amino acid sequence set forth in SEQ ID NO: 63 or a variant sequence thereof, and CDRL3 of the amino acid sequence set forth in SEQ ID NO: 64 or a variant sequence thereof. In a specific embodiment, the antibody can be an antibody, or a variant thereof, comprising (a) a VH having the amino acid sequence set forth in SEQ ID NO: 46 or a variant sequence thereof and a VL having the amino acid sequence set forth in SEQ ID NO: 48 or a variant sequence thereof, or (b) a VH having the amino acid sequence set forth in SEQ ID NO: 50 or a variant sequence thereof and a VL having the amino acid sequence set forth in SEQ ID NO: 52 or a variant sequence thereof. A variant sequence preferably does not comprise an alternation within the CDRs (amino acid substitution, addition, or deletion), but can comprise several alterations (3 or less, 2 or less, preferably 1 or less) within each CDR, as long as the ability to bind and/or function of an antibody before the alteration is maintained. Preferably, a variant sequence can have at least 90% identity to the original sequence.

In another aspect, the antibody of the present disclosure relates to an antibody having a VH with the amino acid sequence set forth in SEQ ID NO: 18 or SEQ ID NO: 22. Preferably, the antibody of the present disclosure has a VH with the amino acid sequence set forth in SEQ ID NO: 18 and a VL with the amino acid sequence set forth in SEQ ID NO: 20, or a VH with the amino acid sequence set forth in SEQ ID NO: 22 and a VL with the amino acid sequence set forth in SEQ ID NO: 24.

Furthermore, the present disclosure relates to an antibody having a VH with an amino acid sequence encoded by a nucleic acid sequence that hybridizes, under a stringent condition, with a nucleic acid sequence encoding the amino acid sequence of SEQ ID NO: 18 or SEQ ID NO: 22. Preferably, the present disclosure relates to an antibody having a VH with an amino acid sequence encoded by a nucleic acid sequence that hybridizes, under a stringent condition, with a nucleic acid sequence encoding the amino acid sequence of SEQ ID NO: 18, and a VL with an amino acid sequence encoded by a nucleic acid sequence that hybridizes, under a stringent condition, with a nucleic acid sequence encoding the amino acid sequence of SEQ ID NO: 20, or an antibody having a VH with an amino acid sequence encoded by a nucleic acid sequence that hybridizes, under a stringent condition, with a nucleic acid sequence encoding the amino acid sequence of SEQ ID NO: 22, and a VL with an amino acid sequence encoded by a nucleic acid sequence that hybridizes, under a stringent condition, with a nucleic acid sequence encoding the amino acid sequence of SEQ ID NO: 24. As used herein, hybridizing under a stringent condition means hybridizing under a hybridization condition that is generally used by those skilled in the art. Whether a sequence hybridizes can be determined by the method described in, for example, Molecular Cloning, a Laboratory Manual, Fourth Edition, Cold Spring Harbor Laboratory Press (2012), Current Protocols in Molecular Biology, Wiley Online Library, or the like. For example, a hybridization condition can be a condition for hybridizing at 42° C. in 6×SSC (0.9M NaCl, 0.09M trisodium citrate) or 6×SSPE (3M NaCl, 0.2M NaH2PO4, 20 mM EDTA·2Na, pH 7.4) and then washing at 42° C. in 0.5×SSC.

The present disclosure also comprises an antibody having a VH with an amino acid sequence with 80% or greater identity to the amino acid sequence of SEQ ID NO: 18 or SEQ ID NO: 22. Preferably, the antibody of the present disclosure has a VH with an amino acid sequence with 80% or greater identity to the amino acid sequence of SEQ ID NO: 18 and a VL with an amino acid sequence with 80% or greater identity to the amino acid sequence of SEQ ID NO: 20, or a VH with 80% or greater identity to the amino acid sequence of SEQ ID NO: 22 and a VL with an amino acid sequence with 80% or greater identity to the amino acid sequence of SEQ ID NO: 24. Identity of amino acid sequences refers to a ratio (%) of the numbers of amino acids of the same type between two types of proteins in the range of amino acid sequence targeted for comparison, and can be determined using a known program such as BLAST or FASTA. The identity described above can be identity that is higher than 80% or greater, such as identity of 85% or greater, 90% or greater, 95% or greater, 98% or greater, or 99% or greater.

An antibody having the VH described above with an amino acid sequence encoded by a nucleic acid sequence that hybridizes under a stringent condition, or an antibody having an amino acid sequence with 80% or greater identity preferably has the CDR sequence described above.

As a more specific example, the present disclosure comprises an antibody having a heavy chain with the amino acid sequence set forth in one SEQ ID NO. selected from SEQ ID NO: 2, SEQ ID NO: 6, SEQ ID NO: 10, and SEQ ID NO: 14. Preferably, the antibody of the present disclosure has a heavy chain with the amino acid sequence set forth in SEQ ID NO: 2 and a light chain with the amino acid sequence set forth in SEQ ID NO: 4, a heavy chain with the amino acid sequence set forth in SEQ ID NO: 6 and a light chain with the amino acid sequence set forth in SEQ ID NO: 8, a heavy chain with the amino acid sequence set forth in SEQ ID NO: 10 and a light chain with the amino acid sequence set forth in SEQ ID NO: 12, or a heavy chain with the amino acid sequence set forth in SEQ ID NO: 14 and a light chain with the amino acid sequence set forth in SEQ ID NO: 16.

In a preferred embodiment, an antibody can comprise (a) a VH comprising CDRH1 of the amino acid sequence set forth in SEQ ID NO: 25, CDRH2 of the amino acid sequence set forth in SEQ ID NO: 26, and CDRH3 of the amino acid sequence set forth in SEQ ID NO: 27, and a VL comprising CDRL1 of the amino acid sequence set forth in SEQ ID NO: 28, CDRL2 of the amino acid sequence set forth in SEQ ID NO: 29, and CDRL3 set forth in SEQ ID NO: 30, or (b) a VH comprising CDRH1 of the amino acid sequence set forth in SEQ ID NO: 31, CDRH2 of the amino acid sequence set forth in SEQ ID NO: 32, and CDRH3 of the amino acid sequence set forth in SEQ ID NO: 33, and a VL comprising CDRL1 of the amino acid sequence set forth in SEQ ID NO: 34, CDRL2 of the amino acid sequence set forth in SEQ ID NO: 35, and CDRL3 set forth in SEQ ID NO: 36. In another preferred embodiment, an antibody can comprise (a) a VH having the amino acid sequence set forth in SEQ ID NO: 18 or a variant sequence thereof, and a VL having the amino acid sequence set forth in SEQ ID NO: 20 or a variant sequence thereof, or (b) a VH having the amino acid sequence set forth in SEQ ID NO: 22 or a variant sequence thereof, and a VL having the amino acid sequence set forth in SEQ ID NO: 24 or a variant sequence thereof. A variant sequence preferably does not comprise an alteration (amino acid substitution, addition, or deletion) within a CDR, but can comprise an alternation of several alternations (3 or less, 2 or less, preferably 1 or less) within each CDR, as long as the ability to bind and/or function of an antibody before the alteration is maintained. Preferably, a variant sequence can have at least 90% identity to the original sequence.

The antibody of the present disclosure can be prepared by immunizing a non-human animal with an immune response, preferably a non-human animal (e.g., MRL/1pr mouse) with an (autoantibody) immune response to itself due to hyperimmunization, with a CD80, CD86, or CD28 protein or an extracellular domain thereof, or a cell expressing CD80, CD86, or CD28 as an immunogen, together with an immunostimulant (e.g., mineral oil or aluminum precipitate and heated dead bacteria or lipopolysaccharide, Freund's complete adjuvant, Freund's incomplete adjuvant, or the like) as needed. An immunized animal is not particularly limited, as long as it is an animal for which a hybridoma can be prepared, such as a mouse, rat, hamster, guinea pig, rabbit, dog, monkey, sheep, goat, chicken, or duck, but is preferably a mouse or a rat, more preferably a mouse, and most preferably an MRL/lpr mouse. An immunogen can be administered to an animal by subcutaneous injection, intraperitoneal injection, intravenous injection, intradermal injection, intramuscular injection, or foot sole injection of 1×10⁶ cells once or several times at a suitable interval (generally, one immunization every 1 to 6 weeks for a total of about 2 to 10 times). After 1 to 2 weeks from the last immunization, blood is collected from the eye socket or caudal vein of the immunized animal to measure the antibody titer using the serum thereof. The antibody of the present disclosure can be obtained by purification from the serum of an animal exhibiting sufficient antibody titer.

A monoclonal antibody can be obtained by culturing a hybridoma obtained by fusing an antibody producing cell obtained from an immunized animal that has been immunized through the method described above with a myeloma cell. Examples of the fusion method include the method of Milstein et al. (Galfre, G. & Milstein, C. (1981) Methods Enzymol., 73:3-46). An antibody producing cell to be used can be harvested from the spleen, pancreas, lymph node, or peripheral blood of a mouse or rat, which is immunized by the method described above and has serum exhibiting sufficient antibody titer. A myeloma cell to be used is not particularly limited, as long as it is a cell that is derived from a mammal such as a mouse, rat, guinea pig, hamster, rabbit, or human and can proliferate in vitro. Examples of such a cell include P3-X63Ag8 (X63) (Nature, 256, 495, 1975), P3/NS1/1-Ag4-1 (NS1) (Eur. J. Immunol., 6, 292, 1976), P3X63Ag8U1 (P3U1) (Curr. Top. Microbiol. Immunol., 81, 1, 1978), P3X63Ag8. 653 (653) (J. Immunol., 123, 1548, 1979), Sp2/0-Ag14 (Sp2/O) (Nature, 276, 269, 1978), Sp2/O/FO-2 (FO-2) (J. Immunol. Methods, 35, 1, 1980), SP2ab, and the like. The cell is preferably a cell derived from an animal of the same species as the antibody producing cell, and more preferably a cell derived from an animal of the same phylogenic tree as the antibody producing cell.

After culture, the culture supernatant is collected, and a clone binding to CD80, CD86, or CD28 is selected through ELISA using a CD80, CD86, or CD28 protein or an extracellular domain thereof, or a cell expressing CD80, CD86, or CD28. A cell producing monoclonal antibody can be obtained by repeating limiting dilution 1 to 5 times on the selected clone to prepare a single cell.

Alternatively, an antibody binding to CD80, CD86, or CD28 can be obtained, for example, by utilizing an antibody phage library (Tomizuka et al., Nature Genet., 15, 146-156 (1997)). When utilizing an antibody phage library, a desired clone can be obtained by, for example, immobilizing a CD80,CD86, or CD28 protein or an extracellular domain thereof, or a cell expressing CD80, CD86, or CD28 to a solid phase, reacting this with a phage library, washing and removing unbound phage, and then collecting the bound phage (panning).

Alternatively, this can be obtained by screening an antibody with high specificity to CD80, CD86, or CD28 in accordance with the method described above from antibodies obtained by designing the amino acid sequence of an antibody of interest or an immune responsive fragment thereof by referring to the amino acid sequence described herein, preparing a DNA encoding the designed amino acid sequence, incorporating the DNA into an expression vector, and introducing and expressing said vector in a suitable host cell.

When the antibody of the present disclosure is a human type chimeric antibody, the antibody can be obtained by preparing a DNA encoding VH and VL of a non-human animal monoclonal antibody, binding this with a constant region cDNA of a human immunoglobulin and integrating this into an expression vector, and introducing and expressing the vector in a suitable host cell (Morrison, S. L. et al., Proc. Natl. Acad. Sci. USA, 81, 6851-6855, 1984).

When the antibody of the present disclosure is a humanized antibody, the antibody can be obtained by constructing a DNA encoding a V region prepared by transplanting an amino acid sequence encoding a CDR of a VH and VL of a non-human animal monoclonal antibody into a Framework Region (FR) of a VH and VL of a human antibody, binding the constructed DNA with a constant region cDNA of a human derived immunoglobulin and integrating this into an expression vector, and introducing and expressing the vector into a suitable host cell (see L. Rieohmann et al., Nature, 332, 323, 1988: Kettleborough, C. A. et al., Protein Eng., 4, 773-783, 1991; Clark M., Immunol. Today., 21, 397-402, 2000). A CDR of a non-human animal monoclonal antibody can be obtained by comparing an amino acid sequence predicted from a DNA sequence encoding a VH and VL of a non-human animal monoclonal antibody obtained by the method described above with the full amino acid sequences of the VH and VL of a known antibody. The amino acid sequences of a known antibody can be obtained from the amino acid sequences of antibodies that are registered in a database such as a protein data bank. An FR of a humanized antibody is not particularly limited as long as an antibody after transplantation attains the effect of the present disclosure, but is preferably an FR of a human antibody with a three-dimensional structure where the variable region (hereinafter, referred to as the “V region”) of the humanized antibody is similar to the V region of a non-human animal monoclonal antibody from which the CDR is derived, or a human antibody FR with high homology to the amino acid sequence of an FR of a non-human animal monoclonal antibody to be used. Some of the amino acids constituting an FR derived from a human antibody (particularly amino acids positioned in the vicinity of a CDR three dimensionally) can be substituted with an FR sequence of a non-human animal monoclonal antibody from which a CDR is derived as needed in a humanized antibody (see Queen et al., U.S. Pat. No. 5,585,089). A DNA sequence encoding a V region of a humanized antibody to be used is designed as a DNA sequence corresponding to an amino acid sequence prepared by binding an amino acid sequence of a CDR of a non-human animal monoclonal antibody with an amino acid sequence of an FR of a human antibody. A DNA encoding a V region of a humanized antibody can be prepared by a method that is well known to those skilled in the art based on a designed DNA sequence.

For example, a human antibody can be obtained by utilizing a human antibody phage library or a human antibody producing transgenic mouse (Tomizuka et al., Nature Genet., 15, 146-156 (1997)). When utilizing a human antibody phage library, a desired clone can be obtained by, for example, binding a CD80, CD86, or CD28 protein or an extracellular domain thereof, or a cell expressing CD80, CD86, or CD28 to a solid phase, reacting this with a phage library, washing and removing unbound phage, and then collecting the bound phage (panning). A human antibody producing transgenic mouse is a mouse produced by introducing an Ig gene of a human antibody into an endogenous immunoglobulin (Ig) gene human antibody that specifically knockout mouse. A recognizes CD80, CD86, or CD28 can be obtained by immunizing an antigen protein in accordance with the antibody preparation method of the present disclosure described above, while using a human antibody producing transgenic mouse as an immunized animal.

### Nucleic Acid Molecule/Vector/Host Cell

In another aspect, the present disclosure relates to a nucleic acid molecule having a polynucleotide encoding the antibody of the present disclosure described above. For example, the nucleic acid molecule of the present disclosure comprises the nucleotide sequence set forth in SEQ ID NO: 17 or SEQ ID NO: 21 as a VH. In another aspect, the nucleic acid molecule of the present disclosure comprises the nucleotide sequence set forth in SEQ ID NO: 19 or SEQ ID NO: 23 as a VL. As a more specific example, the nucleic acid molecule of the present disclosure comprises a polynucleotide encoding a VH having the nucleotide sequence set forth in any one selected from SEQ ID NOs: 1, 5, 9, and 13 and a polynucleotide encoding a VL with the nucleotide sequence set forth in any one selected from SEQ ID NOs: 3, 7, 11, and 15. The nucleic acid molecule of the present disclosure can have a nucleic acid sequence that hybridizes under a stringent condition with the SEQ ID NO. described above. Furthermore, the present disclosure encompasses a vector having said nucleic acid molecule. Such a vector is not particularly limited, as long as it is a vector that can be utilized in the expression of an antibody. A suitable viral vector, a plasmid vector, or the like can be selected in accordance with the host that is used. In another aspect, the present disclosure relates to a host cell comprising said vector. The host cell is not particularly limited as long as it is a host cell that can be utilized in the expression of an antibody. Examples thereof include mammalian cells (mouse cells, rat cells, rabbit cells, human cells, and the like) yeast, and microorganisms (E. coli and the like). The present disclosure comprises a method of manufacturing the antibody of the present disclosure, comprising culturing the host cell described above. A medium culturing method can be appropriately determined by well-known means of those skilled in the art in accordance with the host cell that is used.

The nucleic acid of the present disclosure can be obtained by cloning from a hybridoma producing an antibody obtained above, or designing an appropriate nucleic acid sequence based on the amino acid sequence of the antibody obtained above or an immune responsive fragment thereof. The vector of the present disclosure can be obtained by incorporating the resulting nucleic acid into a vector suitable for expression as appropriate. The vector of the present disclosure can comprise a region that is required for expression (promotor, enhancer, terminator, or the like) in addition to the nucleic acid of the present disclosure. The host cell of the present disclosure can be obtained by introducing the vector of the present disclosure into a suitable cell line (e.g., animal cell, insect cell, plant cell, yeast, or microorganism such as E. coli).

### Composition

In one embodiment of the present disclosure, the present disclosure provides a composition for preparing a cell having immune tolerance induced, comprising at least one of the antibodies described herein. In one embodiment of the present disclosure, a composition can comprise an anti-CD80 antibody, an anti-CD86 antibody, an anti-CD28 antibody, a bispecific antibody to CD80 and CD86, or any combination thereof. Rejection due to organ transplantation can be reduced by, for example, administering to cell having immune tolerance induced to a subject receiving organ transplantation. A cell having immune tolerance induced can be a cell derived from a subject (recipient) or a donor, or a mixture thereof. The composition of the present disclosure surprisingly does not substantially induce release of cytokines due to a cytokine producing cell (e.g., macrophage, neutrophil, or natural killer (NK) cell). Since cytokines attenuate immune tolerance, it is advantageous that release of cytokines is not induced.

In another aspect, the present disclosure relates to a medical composition comprising the antibody described herein. In one example, the medical composition of the present disclosure comprises an anti-CD80 antibody, an anti-CD86 antibody, an anti-CD28 antibody, or a bispecific antibody to CD80 and CD86. The medical composition of the present disclosure can be used to induce immune tolerance. Throughout the entire specification, “to induce immune tolerance” includes cases of use, for example, to induce immune tolerance to a transplanted organ, i.e., to suppress excessive immune responses to a causative agent that becomes an immunogen in a patient with an autoimmune disease or allergic disease to alleviate or treat a symptom of such diseases in order to suppress rejection in organ transplantation. In another embodiment, the pharmaceutical composition of the present disclosure is used to prepare a cell having immune tolerance induced.

As used herein, “organ” to be transplanted in “organ transplantation” is not particularly limited. Examples thereof include a heart, kidney, lung, pancreas, esophagus, stomach, small intestine, large intestine, skin, nerve, blood, blood cells including immune system cells, bone, cartilage, blood vessel, cornea, eye ball, bone marrow, and liver. As used herein, “organ” includes a part of an organ and cells constituting an organ in addition to the entirety or a mass of an organ. For example, transplanted organs also include myocardial cells, corneal cells, and the like. Organ transplantation also includes transplantation of an organ made using heterologous stem cells in regenerative medicine or the like into a patient in addition to transplantation of an organ of another individual into a patient.

The antibody of the present disclosure can be used as a medical composition by purifying and then formulating in accordance with a conventional method as needed. The present disclosure also comprises use of the antibody of the present disclosure for the manufacture of a medical composition for inducing immune tolerance. Alternatively, the present disclosure comprises use of the antibody of the present disclosure for inducing immune tolerance. Furthermore, the present disclosure relates to a method of inducing immune tolerance, comprising adding or administering the antibody of the present disclosure. For example, the present disclosure comprises a method of suppressing rejection in organ transplantation, comprising administering the antibody of the present disclosure to a patient receiving organ transplantation, a method of treating an autoimmune disease or allergic disease, comprising administering the antibody of the present disclosure to a patient with an autoimmune disease or allergic disease, and a method of suppressing rejection in transplantation of an iPS cell or ES cell or a cell, tissue, or organ derived (differentiated) therefrom, comprising administering antibody of the present disclosure to a patient receiving transplantation of an iPS cell or ES cell or a cell, tissue, or organ derived (differentiated) therefrom.

Examples of cells, tissue, or organ derived (differentiated) from iPS cells or ES cells include, but are not limited to, nervous cells or tissue, corneal cells or tissue, myocardial cells or tissue, liver cells or tissue, cartilage cells or tissue, skin cells or tissue, kidney cells or tissue, and the like. In a preferred embodiment, cells, tissue, or organ derived (differentiated) from iPS cells or ES cells include nervous cells or tissue, myocardial cells or tissue, cartilage cells or tissue, and skin cells or tissue.

The medical composition of the present disclosure can be used through direct administration to a patient, or through contacting the composition with a PBMC harvested from a patient whose immune tolerance is to be induced, together with an antigen of interest inducing immune tolerance ex vivo to prepare an immune cell that is anergic to the antigen.

When the medical composition of the present disclosure is directly administered to a patient, any oral or parenteral formulation can be used. Examples of a composition for parenteral administration include eye drops, injections, nasal drops, suppositories, patches, ointments, and the like. The composition is preferably an injection. Examples of the dosage forms of the medical composition of the present disclosure include a liquid agent and a lyophilized formulation. When the medical composition of the present disclosure is used as an injection, an additive, e.g., a solubilizing agent such as propylene glycol or ethylenediamine, buffer such as phosphate, tonicity agent such as sodium chloride or glycerine, stabilizer such as sulfite, preservative such as phenol, or analgesic such as lidocaine, can be added as needed (see “Japanese Pharmaceutical Excipients Directory”, Yakuji Nippo, Limited, “Handbook of Pharmaceutical Excipients Fifth Edition” APhA Publications). When the medical composition of the present disclosure is used as an injection, examples of a storage container include an ampule, vial, prefilled syringe, pen-shaped injection cartridge, IV bag, and the like.

For example, the medical composition of the present disclosure (therapeutic drug or preventive drug) can be utilized as an injection. The dosage form thereof includes intravenous injection, subcutaneous injection, intradermal injection, intramuscular injection, intravitreous injection, intravenous drip injection, and the like. Such injections can be prepared in accordance with a known method, for example, by dissolving, suspending, or emulsifying the antibody described above in an aseptic aqueous or oily solution that is generally used in injection. For example, tonicity solution comprising saline, glucose, sucrose, mannitol, or other adjuvant or the like can be used as an aqueous solution for injection, and can be used in conjunction with a suitable solubilizing agent such as alcohol (e.g., ethanol), polyalcohol (e.g., propylene glycol or polyethylene glycol), anionic surfactant [e.g., polysorbate 80, polysorbate 20, HCO-50 (polyoxyethylene (50 mol) adduct of hydrogenated castor oil)] or the like. For example, sesame oil, soybean oil or the like can be used as an oily solution, which may be used concomitantly with benzyl benzoate, benzyl alcohol, or the like as a solubilizing agent. The prepared injection is generally filled in a suitable ampule, vial, or syringe. A suitable excipient can be added to the antibody of the present disclosure or an immune responsive fragment thereof to prepare a lyophilized formulation, which is dissolved in injection water, saline, or the like when needed as an injection solution. While oral injection of a protein such as an antibody is generally considered difficult due to decomposition by digestive organs, oral administration may be possible by creativity and ingenuity in the dosage form, such as an antibody fragment or modified antibody fragment. Examples of orally administered formulations include capsules, tablets, syrup, granules, and the like.

The medical composition of the present disclosure is suitably prepared into a dosage form in a dosing unit that matches the dosage of an active ingredient. Exemplary dosage form in a dosing unit includes an injection (ampule, vial, or prefilled syringe). Generally, 5 to 500 mg, 5 to 100 mg, or 10 to 250 mg of the antibody of the present disclosure or an immune responsive fragment thereof can be contained per dosage form in a dosing unit.

Administration of the medical composition of the present disclosure can be topical or systemic. The administration method is not particularly limited. The composition is administered parenterally or orally as described above. Examples of parenteral routes of administration include intraocular, subcutaneous, intraperitoneal, blood (intravenous or intra-arterial), or spinal fluid injection, drop, and the like, and is preferably administration into the blood. The medical composition of the present disclosure (therapeutic drug or preventive drug) can be temporarily administered, or continuously or intermittently administered. For example, the administration can be continuous administration for 1 minute to 2 weeks.

The dosage of the medical composition of the present disclosure is not particularly limited, as long as it is a dosage attaining a desired therapeutic or preventive effect. The dosage can be appropriately determined depending on the symptom, sex, age, or the like. The dosage of the medical composition of the present disclosure can be determined, for example, with the degree of induction of immune tolerance as an indicator. For example, as a single dose of an active ingredient of the medical composition of the present disclosure, it is advantageous to administer generally about 0.01 to 20 mg/kg body weight, preferably about 0.1 to 10 mg/kg body weight, and still more preferably about 0.1 to 5 mg/kg body weight about 1 to 10 times a month and preferably about 1 to 5 times a month by intravenous injection. An amount corresponding thereto can be administered for other parenteral or oral administration. If the symptom is particularly severe, the amount or the number of administrations can be increased in accordance with the symptom.

When the medical composition of the present disclosure is contacted with a PBMC harvested from a patient whose immune tolerance is to be induced, together with an antigen of interest inducing immune tolerance ex vivo to prepare an immune cell that is anergic to the antigen, the composition can be prepared in a form such as liquid, powder, tablet, gel, or granule in accordance with the composition for administration described above.

### Immune Tolerance Induced Cells

In another aspect, the present disclosure provides a cell having immune tolerance induced by the antibody described herein. Immune tolerance can be induced by mixing a cell derived from a subject with an antigen that is not derived from the subject or an antigen containing material. In still another aspect of the present disclosure, the present disclosure provides a method for manufacturing a cell for treating or preventing a disease, disorder, or condition caused by an antigen that is not derived from a subject, comprising mixing the antibody described herein, a cell derived from a subject, and an antigen that is not derived from the subject or an antigen containing material (e.g., cell). The disease, disorder, or condition can be selected from the group consisting of graft rejection, allergy, autoimmune disease, graft-versus-host disease, and immune rejection caused by transplantation of an iPS cell or an ES cell and a cell, tissue, or organ derived from said cells.

In an embodiment where the disease or the like targeted by the present disclosure is graft rejection, an anergic cell can be induced by mixing the antibody described above with a recipient derived cell (PBMC or splenocyte) and a donor derived antigen or a donor derived antigen containing material. A donor derived antigen containing material can be a PBMC, splenocyte, or a cell derived from an organ to be transplanted.

In an embodiment where the disease targeted by the present disclosure or the like is allergy, an anergic cell can in induced by mixing the antibody described above with a subject derived cell (PBMC or splenocyte) and an allergy inducing antigen that is not derived from the subject.

In an embodiment where the disease targeted by the present disclosure or the like is an autoimmune disease, an anergic cell can in induced by mixing the antibody described above with a subject derived cell (PBMC or splenocyte) and a subject derived antigen that is the cause of the autoimmune disease.

In an embodiment where the disease targeted by the present disclosure or the like is a graft-versus-host disease, an anergic cell can be induced by mixing the antibody described above with a PBMC or splenocyte of a donor providing the graft and recipient derived antigen or a material containing the antigen. The material containing an antigen derived from a recipient can be a PBMC, splenocyte, a cell around a site where an organ is transplanted, or a cell derived therefrom.

In an embodiment where the disease targeted by the present disclosure or the like is immune rejection caused by transplantation of an iPS cell or an ES cell and a cell, tissue, or organ derived from said cells, an anergic cell can be induced by mixing the antibody described above with a subject derived cell (PBMC or splenocyte) and a cell differentiated from an iPS cell or ES cell used in transplantation.

Therapeutic examples of a disease or the like according to the present disclosure are shown below, but are not limited thereto.

### Allergy and Autoimmune Disease

For allergy and autoimmune diseases, a macrophage obtained from the peripheral blood of a patient is differentiated into a dendritic cell (macrophage derived dendritic cell) with high antigen presenting ability by a conventional method. The cell after irradiation of radiation (γ rays) is made to present an antigen that is the cause of hyper-reaction in allergy or autoimmune disease, and co-cultured for 1 to 2 weeks with a T cell group contained form the same patient peripheral blood in the presence of an anti-CD80 antibody and/or anti-CD86 antibody to obtain an anergic cell that is specific to the antigen causing the allergy or autoimmune disease. The anergic cell is administered to a patient to induce immune tolerance specific to the antigen causing the allergy or autoimmune disease for use in the prevention or treatment of allergy and autoimmune disease. The number of dosings can be multiple, depending on whether the dosing is preventive therapy or treatment, and various conditions such as the severity of symptoms.

### Graft-Versus-Host Disease

For graft-versus-host disease, in contrast to the treatment of graft rejection, a cell that can be the cause of graft-versus-host disease such as a PBMC or T cell of a donor providing a graft is co-cultured for 1 to 2 weeks with a PBMC derived from a host irradiated with radiation (γ rays) or other cells in the presence of an anti-CD80 antibody and/or anti-CD86 antibody to obtain an anergic cell that is specific to a host. Administration of such an anergic cell to a host suppresses responses to a host by a graft causing the graft-versus-host disease (and induces immune tolerance) to prevent or treat graft-versus-host disease. The number of dosings can be multiple, depending on whether the dosing is preventive therapy or treatment, and various conditions such as the tissue to be transplanted, the size thereof, or the severity of symptoms.

### Immune Rejection Caused by Transplantation of an iPS Cell or an ES Cell and a cell, TISSUE, or Organ Derived From Said Cells

In applications to treatment using an iPS cell or ES cell, a dendritic cell or a cell used in transplantation differentiated from an iPS cell or ES cell is irradiated with radiation (γ rays), and the cell is co-cultured for 1 to 2 weeks with a PBMC or T cell group of a patient receiving transplantation in the presence of an anti-CD80 antibody and/or anti-CD86 antibody to obtain an anergic cell that is specific to a cell differentiated from an iPS cell or ES cell. Administration of such an anergic cell to a host induces immune tolerance that is specific to an iPS cell or ES cell derived transplanted cell, tissue, and organ and prevent and treat rejection thereto. The number of dosings can be multiple, depending on whether the dosing is preventive therapy or treatment, and various conditions such as the tissue to be transplanted, the size thereof, or the severity of symptoms.

In a specific embodiment, the present disclosure relates to a method of inducing anergy of a PBMC of a patient to an antigen, comprising contacting the antibody described above (including a composition comprising the antibody described above; the same applies hereinafter) with an antigen to which immune tolerance is to be induced or a cell having said antigen on a surface, and a PBMC. The method of inducing immune tolerance of the present disclosure is performed ex vivo. If, for example, suppression of rejection against a transplanted organ of a patient receiving transplantation is intended in organ transplantation, a PBMC is a cell harvested from the patient receiving the organ transplantation. If a PBMC is intended to treat or improve an autoimmune disease or allergic disease, the PBMC is a cell harvested from the patient.

For example, immune tolerance to suppress rejection of organ transplantation can be induced by the following method. PBMC of a patient receiving A (recipient) organ transplantation and a PBMC of a subject (donor) providing an organ are prepared, and the donor PBMC is irradiated with 30 Gy radiation. The donor PBMC is mixed with the recipient PBMC at 1:1, and the antibody of the present disclosure (preferably a combination of an anti-human CD80 antibody and anti-human CD86 antibody) is added. A recipient PBMC that is anergic to the donor can be prepared by culturing for 3 to 7 days in a 37° C. and 5% CO₂ incubator in a medium for PBMCs.

For example, immune tolerance to treat or improve an autoimmune disease or allergic disease can also be induced by the following method. A patient PBMC is prepared and mixed with an antigen causing an autoimmune disease or allergic disease, and the antibody of the present disclosure (preferably a combination of an anti-human CD80 antibody and anti-human CD86 antibody) is added. A recipient PBMC that is anergic to the antigen can be prepared by culturing for 3 to 7 days in a 37° C. and 5% CO₂ incubator in a medium for PBMCs.

The present disclosure comprises a cell (e.g., PBMC) having anergy induced by the method described above. An anergy induced cell (e.g., PBMC) can induce immune tolerance in a patient by administration to a patient, so that an anergy induced cell (e.g., PBMC) can be used as a cell therapy agent. Accordingly, the present disclosure relates to a cell therapy agent comprising a cell (e.g., PBMC) with anergy induced by the method described above as an active ingredient, preferably a cell therapy agent for suppressing rejection in organ transplantation, comprising a cell (e.g., PBMC) of a patient receiving transplantation with anergy to a transplanted organ induced as an active ingredient, or a cell therapy agent for treating an autoimmune disease or allergic disease, comprising a cell (e.g., PBMC) with anergy to an antigen causing an autoimmune disease or allergic disease due to hyperactive immune response induced as an active ingredient, or a cell therapy agent for suppressing rejection in transplantation of an iPS cell or an ES cell and a cell, tissue, or organ derived from said cells. Exemplary rejection includes graft-versus-host disease.

The cell therapy agent of the present disclosure can induce immune tolerance in a patient by administration to the patient. For example, the present disclosure comprises a method of suppressing rejection in organ transplantation, comprising administering a cell therapy agent for suppressing rejection in organ transplantation, comprising a cell (e.g., PBMC) of a patient receiving transplantation with anergy to a transplanted organ induced as an active ingredient to the patient receiving organ transplantation. The present disclosure also comprises a method of treating an autoimmune disease or allergic disease, comprising administering a cell therapy agent for treating an autoimmune disease or allergic disease, comprising a cell (e.g., PBMC) with anergy to an antigen causing an autoimmune disease or allergic disease due to hyperactive immune response induced as an active ingredient to a patient with an autoimmune disease or allergic disease. The present disclosure also comprises a method of suppressing rejection in transplantation of an iPS cell or an ES cell or a cell, tissue, or organ derived from said cells, comprising administering a cell therapy agent for suppressing rejection in transplantation of an iPS cell or an ES cell or a cell, tissue, or organ derived from said cells, comprising a cell (e.g., PBMC) of a patient receiving transplantation with anergy to an iPS cell or an ES cell or a cell, tissue, or organ derived from said cells to be transplanted as an active ingredient to a patient receiving organ transplantation.

The cell therapy agent of the present disclosure comprises a PBMC in a serum containing/free-medium or saline that can be administered to human. When cells are adsorbent, cells can be supported and stored on a detachable substrate and used by detaching the cells upon use. For example, a substrate from which cells are detachable from a change in temperature (RepCell, CellSeed Inc. Japan) or the like can be used.

The present disclosure can be a method of inducing immune tolerance in a patient, comprising contacting the antibody described above with an antigen for which immune tolerance is to be induced or a cell having the antigen on a surface, and a cell (e.g., PBMC) to induce anergy to the antigen of the cell (e.g., PBMC) of the patient, and administering the anergy induced cell (e.g., PBMC) to the patient. For example, the present disclosure comprises a method of suppressing rejection in organ transplantation, comprising preparing a cell (e.g., PBMC) derived from a patient with immune tolerance to a transplanted organ induced by inducing anergy to the transplanted organ of a cell (e.g., PBMC) of a patient by contacting a donor derived cell with the cell (e.g., PBMC) of the patient, and administering the prepared cell (e.g., PBMC) to a patient receiving organ transplantation. The present disclosure also can be a method of treating or improving an autoimmune disease or an allergic disease, comprising preparing a cell (e.g., PBMC) derived from a patient with immune tolerance to an antigen causing an autoimmune disease or an allergic disease induced by inducing anergy to the antigen of a cell (e.g., PBMC) of a patient by contacting the antigen causing an autoimmune disease or an allergic disease with the cell (e.g., PBMC) of the patient, and administering the prepared cell (e.g., PBMC) to a patient of an autoimmune disease or an allergic disease. The present disclosure also comprises a method of suppressing rejection in transplantation of an iPS cell or an ES cell or a cell, tissue, or organ derived from said cells, comprising preparing a cell (e.g., PBMC) derived from a patient with immune tolerance to an iPS cell or an ES cell or a cell, tissue, or organ derived from said cells to be transplanted induced by inducing anergy to the iPS cell or an ES cell or a cell, tissue, or organ derived from said cells of a cell (e.g., PBMC) of a patient by contacting the iPS cell or an ES cell or a cell, tissue, or organ derived from said cells with the cell (e.g., PBMC) of the patient, and administering the prepared cell (e.g., PBMC) to a patient receiving transplantation of the iPS cell or an ES cell or a cell, tissue, or organ derived from said cells.

Administration of a cell therapy agent (anergy induced cell, such as PBMC) is not particularly limited herein as long as the dosage attains a desired therapeutic or preventive effect and can be appropriately determined depending on the symptom, sex, age, or the like. The dosage of the cell therapy agent of the present disclosure can be determined, for example, by using the degree of induction of immune tolerance as an indicator. For example, a single dose of anergy induced PBMCs of the cell therapy agent of the present disclosure is advantageously administered through intravenous injection at generally about 1.0 to 3.7×10⁷ cells/kg body weight, preferably about 2.0 to 5.0×10⁷ cells/kg body weight, and more preferably about 3.0 to 5.0×10⁷ cells/kg body weight, about 1 to 10 times a month and preferably 1 to 5 times a month. If an immune suppression effect is not observed, the amount or number of administration can be increased depending on the symptom.

### Note

As used herein, “or” is used when “at least one or more” of the listed matters in the sentence can be employed. When explicitly described herein as “within the range of two values”, the range also includes the two values themselves.

Reference literatures such as scientific literatures, patents, and patent applications cited herein are incorporated herein by reference to the same extent that the entirety of each document is specifically described.

The present disclosure has been described while showing preferred embodiments to facilitate understanding. While the present disclosure is described hereinafter based on the Examples, the above descriptions and the following Examples are provided for the sole purpose of exemplification, not limitation of the present disclosure. Thus, the scope of the present disclosure is not limited to the embodiments and Examples that are specifically described herein and is limited only by the scope of claims.

### EXAMPLES

The present disclosure is more specifically described hereinafter based on the Examples. However, the present disclosure is not limited to the Examples. Throughout the entire application, all of the cited references are directly incorporated herein by reference.

The Examples are described hereinafter. The organisms used in the following Examples were handled in compliance with the guidelines specified by the Juntendo University and regulatory agencies. Animal experiments were conducted in compliance with the Juntendo University Animal Experiment Control Rules that have been enacted based on “Act on Welfare and Management of Animals”, “Guideline for Raising and Keeping of Laboratory Animals” (2006 Ministry of the Environment Notification No. 88), and “Fundamental guidelines for proper conduct of animal experiment and related activities in Academic research institutions” (2006 Ministry of Education, Culture, Sports, Science and Technology Notification No. 71). The experiments were planned based on the so-called 3R. A plan has been already approved and accepted after submission thereof and review by the animal experiment committee at the Juntendo University. Use of genetically engineered mice has been approved after examination of the experimental plan for using recombinant DNA producing animals by the university safety committee. For recombinant DNA experiments, a plan has been already approved and accepted after submission thereof and review by the DNA safety committee at the Juntendo University, and studies were conducted in accordance with the relevant regulations such as “Law Concerning the Conservation and Sustainable Use of Biological Diversity through Regulations on the Use of Living Modified Organisms”. Experiment using human peripheral blood (PBMC) was examined based on the principle of fair review and information disclosure and is already approved as being suitable by the ethics committee at the Juntendo University. The studies were conducted while strictly protecting personal information and maintaining anonymity after obtaining information consent for the purpose/plan/method of the studies from specimen providers. Experiments were conducted while complying with the ethical guidelines specified by the ethics committees of the Juntendo University School of Medicine and Sagamihara National Hospital and “guidelines for clinical studies” specified by the Ministry of Health, Labour and Welfare. For reagents, the specific products described in the Examples were used. However, the reagents can be substituted with an equivalent product from another manufacturer (Sigma-Aldrich, Wako Pure Chemical, Nacalai Tesque, R & D Systems, USCN Life Science INC, BD bioscience, BioLegend, or the like).

### (Example 1) Mixed Lymphocyte Reaction (MLR) in the Presence of Anti-Human CD80 and Anti-CD86 Antibodies (Subclass IgG1)

### Materials and Methods

Mononuclear cells (PBMC) were separated from human peripheral blood of three volunteers (one was designated as a donor (donor C), and two were designated as recipients (recipient A and recipient B)) by using Promo cell's Lymphocyte separation Media (Cat. No. C-44010), and adjusted so that the concentration would be 4×10⁶ cells/ml in Biowest's 2% human serum type AB (pool) containing ALyS505N-0 medium (Cell Science & Technology Institute (CSTI) 1020P10). 30 Gy radiation (γ rays) was irradiated onto the donor PBMCs.

Donor PBMCs and recipient PBMCs were dispensed into 4wells of a 96-well plate (Corning, Cat. No. 3799) at 100 μL/well each so that the ratio would be 1:1 (final total of 200 μL/well). A combination of a humanized anti-human CD80 antibody (v2 (subclass IgG1)) and anti-human CD86 antibody (v5 (subclass IgG1)) or a combination of eBioscience's mouse anti-human CD80 antibody (Cat. No. 16-0809-85) (subclass IgG1) and mouse anti-human CD86 antibody (Cat. No. 16-0869-85) (subclass IgG2) was added thereto at 10 μl so that the concentration after adding each antibody would be 0.1, 1, or 10 μg/mL, and culture was started in a 37° C. 5% CO₂ incubator. On day 4 from starting the culture, ³H-thymidine (10 μL) was added. On day 5 from starting the culture (after 16 to 20 hours from addition of ³H-thymidine), cultured cells were harvested with a Cell Harvester (Molecular Devices). The amount of ³H-thymidine incorporation was measured with a scintillation counter.

For IFNγ production, a PBMC mixture that has been mixed by the same method described above with addition of each antibody in the same manner was dispensed into 3 wells of a 48-well Plate (Corning, Cat. No. 3548) at 500 μL/well, and culture was started in a 37° C. 5% CO₂ incubator. On day 6 of culture, supernatant was collected. The collected sample was diluted 10 to 25-fold, and then measurement was taken using Biolegend's Human IFNγ ELISA MAX™ Deluxe, Cat. 430104.

### Results

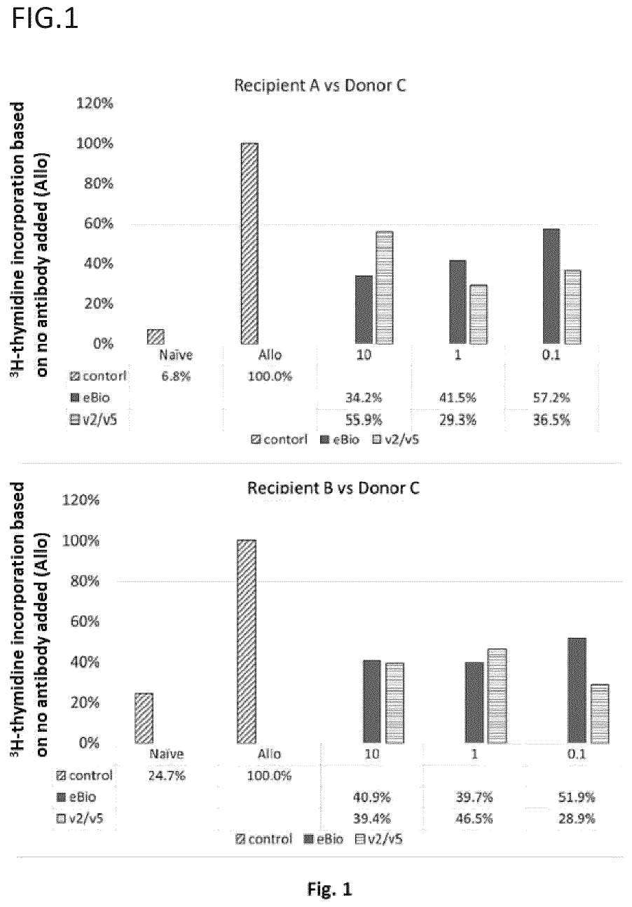

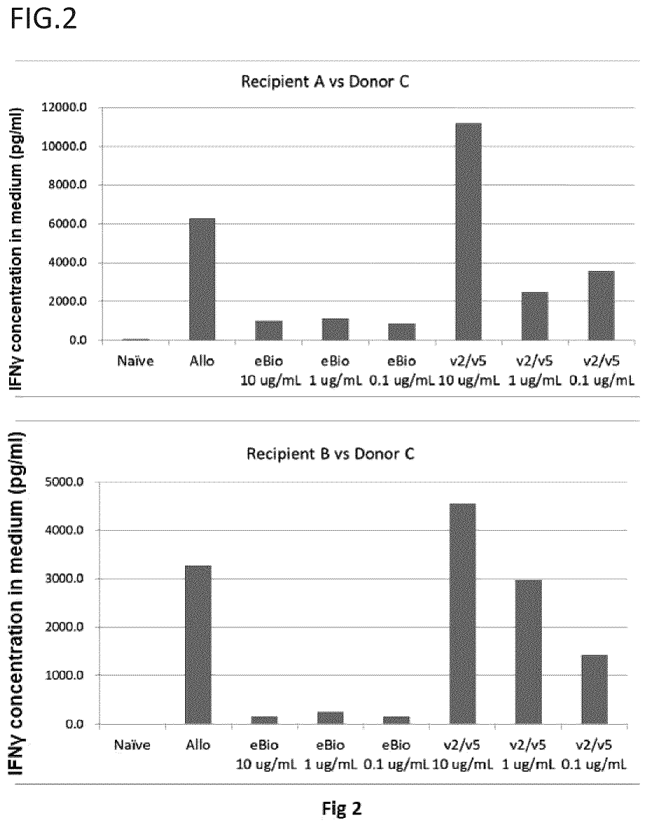

As a result, as shown in FIG. 1, the combination of a humanized anti-human CD80 antibody (v2 (subclass IgG1)) and humanized anti-human CD86 antibody (v5 (subclass IgG1)) had low ³H-thymidine incorporation at nearly the same level as the combination of eBioscience's mouse anti-human CD80 antibody and mouse anti-human CD86 antibody, i.e., suppressed activation of immune cells. Meanwhile, as shown in FIG. 2, IFNγ production evaluation resulted in low production in the presence of eBioscience's mouse anti-human CD80 and CD86 antibodies, whereas IFNγ production dependent on the amount of antibodies added was observed in the presence of a humanized anti-human CD80 and CD86 antibodies (subclass IgG1). At 10 μg/mL, production equal to or greater than that in the absence of antibodies (FIG. 2: Allo) was observed. Although not wishing to be bound by any theory, it is understood that eBioscience's antibodies have low IFNγ production due to being mouse antibodies and having low reactivity.

### (Example 2) Mixed Lymphocyte Reaction (MLR) in the Presence of Anti-Human CD80 and Anti-CD86 Antibodies (Subclass IgG1)

### Materials and Methods

³H-thymidine incorporation when a combination of a humanized anti-human CD80 antibody (v2 (subclass IgG1)) and anti-human CD86 antibody (v5 or v9 (subclass IgG1))) or a combination of eBioscience's mouse anti-human CD80 antibody (Cat. No. 16-0809-85) and mouse anti-human CD86 antibody (Cat. No. 16-0869-85) was added so that the concentration after addition of each antibody would be 10 μg/mL was evaluated by the same method as Example 1. Of the three volunteers, one was designated as a donor (donor F), and two were designated as recipients (recipient D and recipient E).

Further, IFNγ production was evaluated for a combination of a humanized anti-human CD80 antibody (v2 (subclass IgG1)) and humanized anti-human CD86 antibody (v9 (subclass IgG1)) and a combination of eBioscience's mouse anti-human CD80-antibody and anti-human CD86 antibody. For the combination of a humanized anti-human CD80 antibody (v2 (subclass IgG1)) and anti-human CD86 antibody (v9 (subclass IgG1)), IFNγ production when radiation irradiated donor PBMCs were not added was also evaluated in addition to the method of Example 1.

### Results

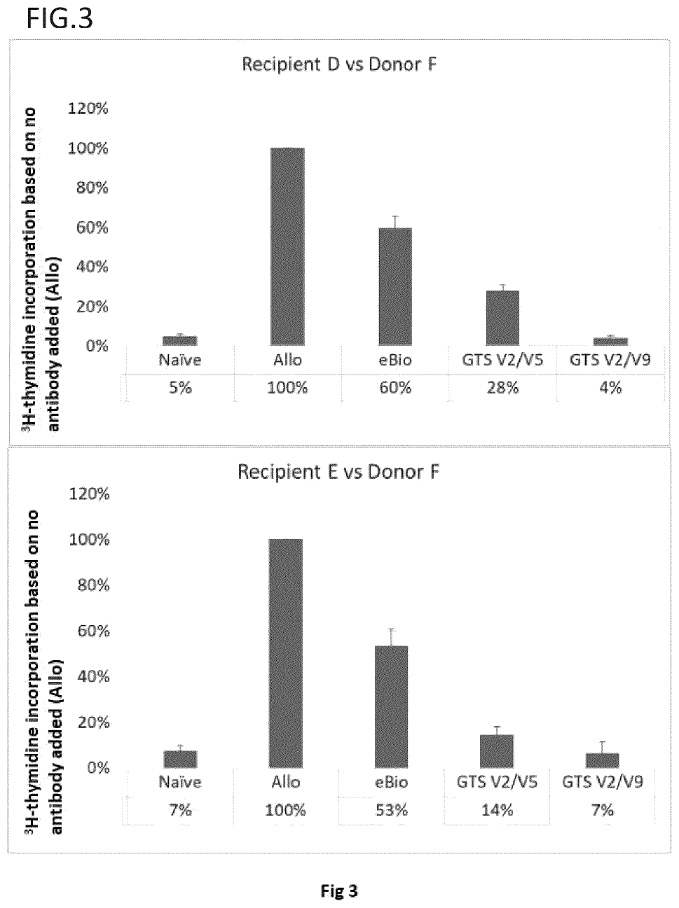

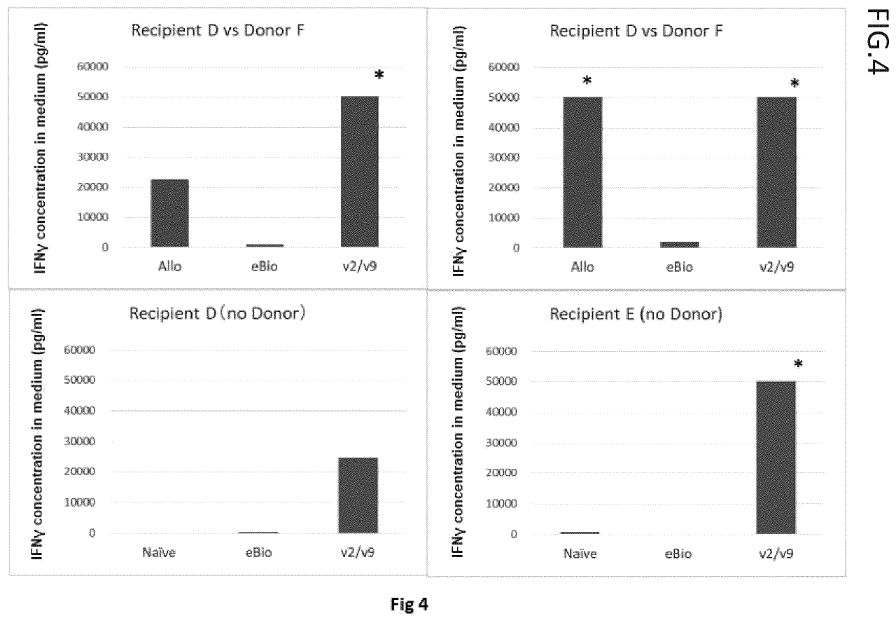

As a result, as shown in FIG. 3, the combination of a humanized anti-human CD80 antibody and anti-human CD86 antibody exhibited greater suppression of ³H-thymidine incorporation than the combination of mouse anti-human CD80-antibody and anti-human CD86 antibody. Meanwhile, as shown in FIG. 4, IFNγ production was low with eBioscience's mouse anti-human CD80 and CD86 antibodies in the same manner as Example 1. Production equal to or greater than that upon donor simulation in the absence of an antibody was observed for humanized anti-human CD80 and CD86 antibodies. Furthermore, the same IFNγ production was observed when humanized anti-CD80 and anti-human CD86 antibodies were added, even radiation irradiated donor PBMCs were not added.

### (Example 3) Mixed Lymphocyte Reaction (MLR) in the Presence of Anti-Human CD80 and Anti-CD86 Antibodies (Subclasses IgG1, IgG2, and IgG4)

In Example 2, IFNγ production was observed even when radiation irradiated donor PBMCs were not added. Thus, the classes of humanized anti-human CD80 antibody and anti-human CD86 antibody were switched, and ³H-thymidine incorporation when a combination of the humanized anti-human CD80 antibody (v2 (subclass IgG2 or IgG4)) and anti-human CD86 antibody (v9 (subclass IgG2 or IgG4)) of the same subclass, and a combination of eBioscience's mouse anti-human CD80 antibody (Cat. No. 16-0809-85) and anti-human CD86 antibody (Cat. No. 16-0869-85) were added so that the concentration after addition of each antibody would be 1, 3, or 10 μg/mL, and IFNγ production when the antibodies were added so that the concentration after addition of each antibody would be 10 μg/mL. Of the three volunteers, one was designated as a donor (donor I), and two were designated as recipients (recipient G and recipient H). The following is the specific experimental system.

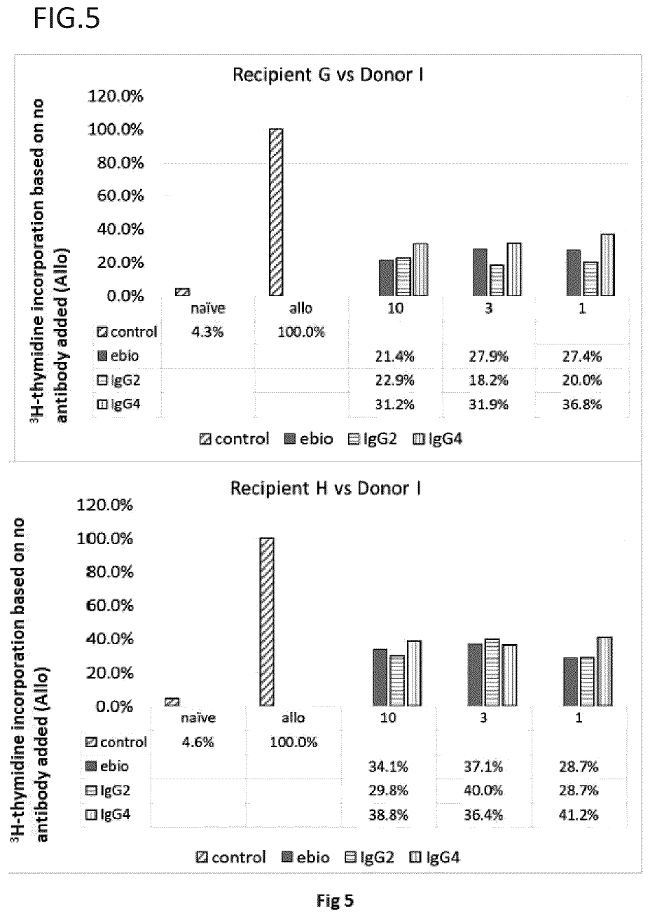

### [³H-thymidine Incorporation (Each Antibody Concentration: 1, 3, and 10 μg/mL) (FIG. 5)

no antibody addition (Allo), radiation irradiated donor PBMC added
eBioscience's mouse anti-human CD80 antibody and mouse anti-human CD86 antibody
humanized anti-human CD80 antibody (v2 (IgG2)) and humanized anti-human CD86 antibody (v9 (IgG2))
humanized anti-human CD80 antibody (v2 (IgG4)) and humanized anti-human CD86 antibody (v9 (IgG4))

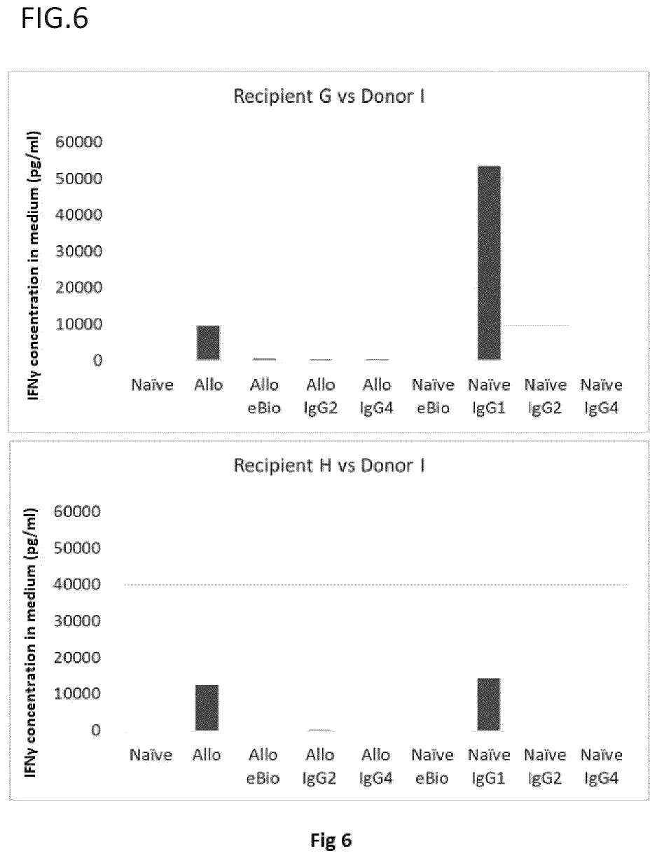

### [IFNγ Production (Each Antibody Concentration: 10 μg/mL) (FIG. 6)

### With Addition of Radiation Irradiated Donor PBMC (Allo)

no antibody addition
eBioscience's mouse anti-human CD80 antibody and anti-human CD86 antibody
humanized anti-human CD80 antibody (v2 (IgG2)) and humanized anti-human CD86 antibody (v9 (IgG2))
humanized anti-human CD80 antibody (v2 (IgG4)) and humanized anti-human CD86 antibody (v9 (IgG4))

### No Addition of Radiation Irradiated Donor PBMC (Naïve)

no antibody addition (Allo)
eBioscience's mouse CD80 antibody and anti-CD86 antibody
humanized anti-CD80 antibody (v2 (IgG1)) and humanized anti-human CD86 antibody (v9 (IgG1))
humanized anti-CD80 antibody (v2 (IgG2)) and humanized anti-human CD86 antibody (v9 (IgG2))
humanized anti-CD80 (IgG4)) and humanized anti-human CD86 antibody (v9 (IgG4))

### Results

As a result, as shown in FIG. 5, subclasses IgG2 and IgG4 exhibited suppression of ³H-thymidine incorporation in the same manner as Examples 1 and 2. Meanwhile, as shown in FIG. 6, IFNγ production was observed in subclass IgG1, but surprisingly IFNγ was not produced at all in subclasses IgG2 and IgG4.

### (Example 4) Mixed Lymphocyte Reaction (MLR) When Cells Obtained From Culturing in the Presence Of Anti-Human CD80 and Anti-Human CD86 Antibodies (Subclass IgG1, IgG2, or IgG4) Were Added

### Materials and Methods

Culture was continued up to day 14 by the method described in Example 1 in the presence of a combination of subclasses IgG1, IgG2, and IgG4 of humanized anti-human CD80 antibody (v2) and anti-human (v9). The humanized anti-human CD80 antibody (v2) of subclass IgG2 consists of a heavy chain with the amino acid sequence of SEQ ID NO: 2 and a light chain with the amino acid sequence of SEQ ID NO: 4. The humanized anti-human CD80 antibody (v2) of subclass IgG4 consists of a heavy chain with the amino acid sequence of SEQ ID NO: 6 and a light chain with the amino acid sequence of SEQ ID NO: 8. The anti-human CD86antibody (v9) of subclass IgG2 consists of a heavy chain with the amino acid sequence of SEQ ID NO: 10 and a light chain with the amino acid sequence of SEQ ID NO: 12. The anti-human CD86 antibody (v9) of subclass IgG4 consists of a heavy chain with the amino acid sequence of SEQ ID NO: 14 and a light chain with the amino acid sequence of SEQ ID NO: 16. After culture, the resulting anergic cells, recipient PBMCs (R-PBMC), and radiation irradiated donor PBMCs (D-PBMC) were mixed in accordance with the following table, and then ³H-thymidine incorporation was evaluated after mixing by the method described in Example 1.

TABLE 1

| Cell suspension | | R-PBMC | Anergic T | D-PBMC | Medium |
|---|---|---|---|---|---|
| Naïve | μL | 200.0 | 0.0 | 0.0 | 300.0 |
| (R-PBMC only) | cells | 4.0 × 10⁵ | 0 | 0.0 | 0 |
| Allo | μL | 200.0 | 0.0 | 200.0 | 100.0 |
| (R-PBMC + D-PBMC) | cells | 4.0 × 10⁵ | 0 | 4.0 × 10⁵ | 0 |
| R-PBMC:Anergic T = | μL | 200.0 | 100.0 | 200.0 | 0.0 |
| 1:1/2 | cells | 4.0 × 10⁵ | 2.0 × 10⁵ | 4.0 × 10⁵ | 0 |
| R-PBMC:Anergic T = | μL | 200.0 | 50.0 | 200.0 | 50.0 |
| 1:1/4 | cells | 4.0 × 10⁵ | 1.0 × 10⁵ | 4.0 × 10⁵ | 0 |
| R-PBMC:Anergic T = | μL | 200.0 | 25.0 | 200.0 | 75.0 |
| 1:1/8 | cells | 4.0 × 10⁵ | 5.0 × 10⁴ | 4.0 × 10⁵ | 0 |

### Results

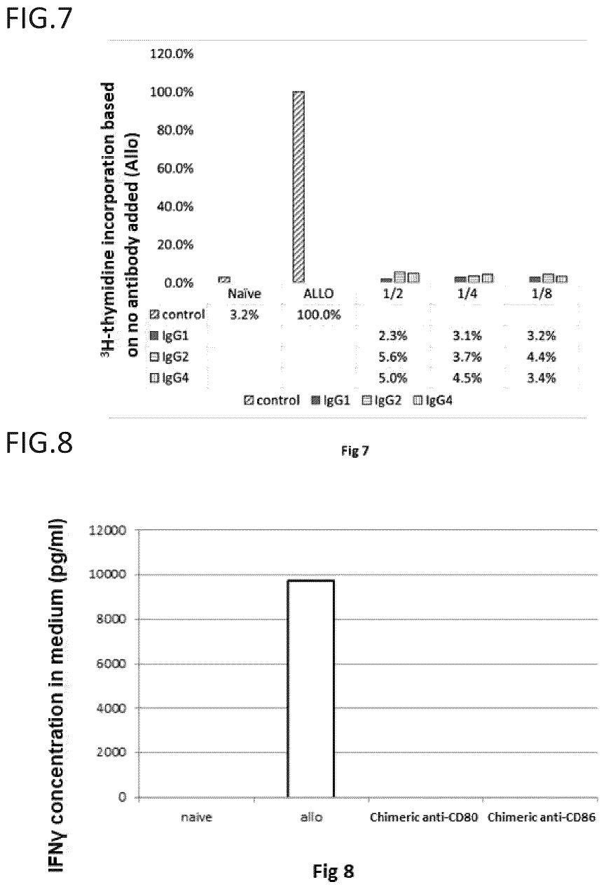

As a result, as shown in FIG. 7, cells obtained in the presence of antibodies of any subclass suppressed ³H-thymidine incorporation equally.

### (Example 5) Induction of Immune Tolerance in Patient Who has Received Organ Transplantation

Tests were conducted by the same method as the method described in Satoru Todo et al. Hepatorogy, 64 (vol. 2), 632-643 (2016). Briefly, anergic T cells that are specific to a donor antigen are induced by co-culturing lymphocytes harvested from the spleen or peripheral blood of a recipient, donor derived lymphocytes irradiated with 30 Gy radiation, and humanized anti-CD80 antibody and anti-CD86 antibody of subclass IgG4 used in Example 4. The anergic T cells are administered on day 13 after surgery to a recipient who has received liver transplantation. The status is subsequently followed up.

### (Example 6) Chimeric Anti-Human CD80 and Chimeric Anti-Human CD86 Antibodies (Subclass IgG1)

### Antibody Preparation

The inventors attempted to improve anti-human CD80 antibodies and anti-human CD86 antibodies for use in cell therapy. Specifically, based on eBioscience's mouse anti-human CD80 antibody and mouse anti-human CD86 antibody, chimeric antibodies of each of them were prepared. The prepared chimeric anti-human CD80 antibody (subclass IgG1) consists of a heavy chain with the amino acid sequence of SEQ ID NO: 38 and a light chain with the amino acid sequence of SEQ ID NO: 40. The prepared chimeric anti-human CD86 antibody (subclass IgG1) consists of a heavy chain with the amino acid sequence of SEQ ID NO: 42 and a light chain with the amino acid sequence of SEQ ID NO: 44. A plasmid encoding them was commissioned to a third party (TPG Biologics, Inc. Taiwan) and expressed from a CHO cell, and antibodies were produced/purified.

### Mixed Lymphocyte Reaction (MLR) in the Presence of Chimeric Anti-Human CD80 and Chimeric Anti-CD86 Antibodies (Subclass IgG1)

### IFNγ production

In almost the same manner as the method described in Example 1, mononuclear cells (PBMC) were separated from human peripheral blood of two volunteers (one was designated as a donor, and the other was designated as a recipient) by using Promo cell's Lymphocyte separation Media (Cat. No. C-44010), and adjusted so that the concentration would be 2×10⁶cells/ml in Biowest's 28 human serum t (pool) containing ALYS505N-0 medium (Cell Science & Technology Institute (CSTI) 1020P10). 30 Gy radiation (γ rays) was irradiated onto the donor PBMCs.

The experimental system was constructed as follows.
1. “naïve”: recipient PBMC, 1×10⁶ cells
2. “allo”: recipient PBMC, 1×10⁶ cells and donor PBMC, 1×10⁶ cells
3. “chimeric anti-CD80”: recipient PBMC, 1×10⁶ cells and chimeric anti-human CD80 antibody, 4 μg
4. “chimeric anti-CD86”: recipient PBMC, 1×10⁶ cells and chimeric anti-human CD86 antibody, 4 μg

Systems 1. to 4. were placed in each well of a 96 well plate (Corning, Cat. No. 3799) so that the final reaction volume would be 200 μL (final concentration of antibodies was about 20 μg/mL), and culture was started in a 37° C. 5% CO₂ incubator. On day 5 and day 6 from starting the culture, supernatant was collected. The collected sample was diluted 20 to 25-fold, and then measurement was taken using Biolegend's Human IFNγ ELISA MAX™ Deluxe, Cat. 430104.

### Results

As shown in FIG. 8, neither the chimeric anti-human CD80 antibody nor the chimeric anti-human CD86 antibody demonstrated detectable IFNγ production.

Measurement of the amount of ³H-thymidine incorporation Basically, experiments were conducted in accordance with the methods described in Examples 1 and 4. Recipient PBMCs and donor PBMCs that were freshly separated from human peripheral blood were used, or those cryopreserved at −80° C. were rapidly thawed and used. These cells were both adjusted so that the concentration would be 4×106 cells/mL in Biowest's 2% human serum type AB (pool) containing ALYS505N-0 medium (Cell Science & Technology Institute (CSTI) 1020P10). 20 Gy radiation was irradiated onto the donor PBMCs. The donor PBMCs and recipient PBMCs were mixed at 1:1, and a chimeric anti-human CD80 antibody and chimeric anti-human CD86 antibody were added to the mixture so that the final concentration would each be 10 μg/ml. The cells were cultured for 7 days in a 37° C. 5% CO₂ incubator on a 6 cm to 10 cm Petri dish (culture volume of 6 to 18 mL).

On day 7 from starting the culture, cultured recipient PBMCs were collected by centrifugation and adjusted to 4×10⁶ cells/mL in the medium described above. To the cultured recipient PBMCs, newly prepared and irradiated donor PBMCs were added so that the cell count ratio would be 2:1, and a chimeric anti-human CD80 antibody and chimeric anti-human CD86 antibody were added so that each would have a final concentration of 5 μg/ml. The cells were cultured for 7 days under the same conditions as above (cell density: 4×10⁶ cells/mL).

On day 14 from starting the culture, induced cells were collected by centrifugation. Each cell was mixed at the following ratio:
1. “naïve”: recipient PBMC, 4×10⁵ cells
2. “allo”: recipient PBMC, 4×10⁵ cells and donor PBMC, 4×10⁵ cells
3. “1:⅛”: recipient PBMC, 4×10⁵ cells, donor PBMC, 4×10⁵ cells, and induced cells 2×10⁵ cells
4. “1:¼”: recipient PBMC, 4×10⁵ cells, donor PBMC, 4×10⁵ cells, and induced cells 1×10⁵ cells
5. “1:⅛”: recipient PBMC, 4×10⁵ cells, donor PBMC, 4×10⁵ cells, and induced cells 5×10⁴ cells
6. “1: 1/16”: recipient PBMC, 4×10⁵ cells, donor PBMC, 4×10⁵ cells, and induced cells 2.5×10⁴ cells

Systems 1. to 6. were each dispensed in 4 wells of a 96 well plate (Corning, Cat. No. 3799) and co-cultured in a 37° C. 5% CO₂ incubator. On day 4 from starting the co-culture, ³H-thymidine (10 μl) was added. On day 5 from starting the co-culture (16 to 20 hours after adding ³H-thymidine incorporation), ³H-thymidine in the culture was removed to measure the amount of ³H-thymidine incorporation.

### Results

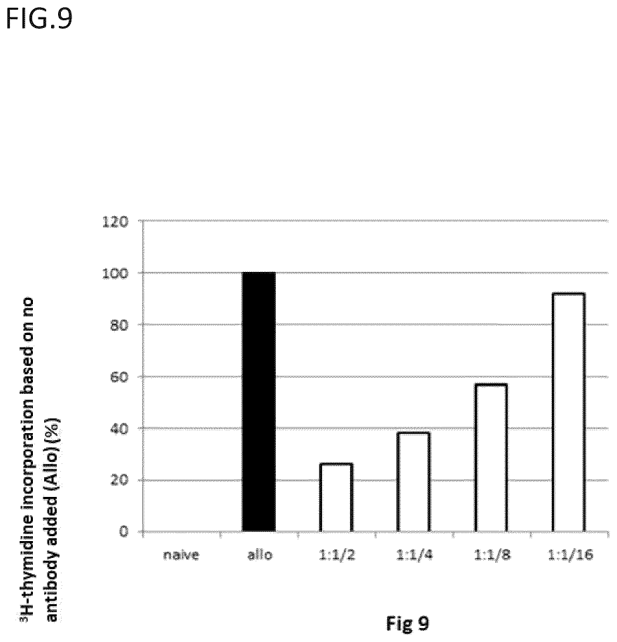

As shown in FIG. 9, induced cells reduced the amount of ³H-thymidine incorporation in a dose dependent manner (i.e., activation of immune cells was suppressed). Therefore, chimeric anti-human CD80 antibodies and chimeric anti-human CD86 antibodies were demonstrated to be antibodies suitable for cell therapy, which can induce anergic T cells without causing detrimental IFNγ production.

### Note

As disclosed above, the present disclosure is exemplified by the use of its preferred embodiments. However, it is understood that the scope of the present disclosure should be interpreted based solely on the Claims. It is also understood that any patent, any patent application, and any other references cited herein should be incorporated herein by reference in the same manner as the contents are specifically described herein. The present application claims priority to Japanese Patent Application No. 2018-118996 (filed on Jun. 22, 2018). It is understood that the content thereof (can be the entire document) is incorporated herein by reference. Further, a part of or the entire content of Japanese Patent Application No. 2018-119001 and Japanese Patent Application No. 2018-1190031 (both filed on Jun. 22, 2018) and international applications claiming priority thereto is incorporated herein by reference.

### INDUSTRIAL APPLICABILITY

The present disclosure provides an antibody with a structure having a feature of improving the effect of immune tolerance or not attenuating the effect of immune tolerance.

A technology that can be utilized in industries (pharmaceutical) related to formulations or the like based on such a technology is provided.

### Sequence Listing Free Text

SEQ ID NO: 1: heavy chain nucleic acid sequence of anti-CD80 antibody (IgG2)
SEQ ID NO: 2: heavy chain amino acid sequence of anti-CD80 antibody (IgG2)
SEQ ID NO: 3: light chain nucleic acid sequence of anti-CD80 antibody (IgG2)
SEQ ID NO: 4: light chain amino acid sequence of anti-CD80 antibody (IgG2)
SEQ ID NO: 5: heavy chain nucleic acid sequence of anti-CD80 antibody (IgG4)
SEQ ID NO: 6: heavy chain amino acid sequence of anti-CD80 antibody (IgG4)
SEQ ID NO: 7: light chain nucleic acid sequence of anti-CD80 antibody (IgG4)
SEQ ID NO: 8: light chain amino acid sequence of anti-CD80 antibody (IgG4)
SEQ ID NO: 9: heavy chain nucleic acid sequence of anti-CD86 antibody (IgG2)
SEQ ID NO: 10: heavy chain amino acid sequence of anti-CD86 antibody (IgG2)
SEQ ID NO: 11: light chain nucleic acid sequence of anti-CD86 antibody (IgG2)
SEQ ID NO: 12: light chain amino acid sequence of anti-CD86 antibody (IgG2)
SEQ ID NO: 13: heavy chain nucleic acid sequence of anti-CD86 antibody (IgG4)
SEQ ID NO: 14: heavy chain amino acid sequence of anti-CD86 antibody (IgG4)
SEQ ID NO: 15: light chain nucleic acid sequence of anti-CD86 antibody (IgG4)
SEQ ID NO: 16: light chain amino acid sequence of anti-CD86 antibody (IgG4)
SEQ ID NO: 17: heavy chain variable region nucleic acid sequence of anti-CD80 antibody (IgG2)
SEQ ID NO: 18: heavy chain variable region amino acid sequence of anti-CD80 antibody (IgG2)
SEQ ID NO: 19: light chain variable region nucleic acid sequence of anti-CD80 antibody (IgG2)
SEQ ID NO: 20: light chain variable region amino acid sequence of anti-CD80 antibody (IgG2)
SEQ ID NO: 21: heavy chain variable region nucleic acid sequence of anti-CD86 antibody (IgG2)
SEQ ID NO: 22: heavy chain variable region amino acid sequence of anti-CD86 antibody (IgG2)
SEQ ID NO: 23: light chain variable region nucleic acid sequence of anti-CD86 antibody (IgG2)
SEQ ID NO: 24: light chain variable region amino acid sequence of anti-CD86 antibody (IgG2)
SEQ ID NO: 25: amino acid sequence of CDRH1 of anti-CD80 antibody (IgG2)
SEQ ID NO: 26: amino acid sequence of CDRH2 of anti-CD80 antibody (IgG2)
SEQ ID NO: 27: amino acid sequence of CDRH3 of anti-CD80 antibody (IgG2)
SEQ ID NO: 28: amino acid sequence of CDRL1 of anti-CD80 antibody (IgG2)
SEQ ID NO: 29: amino acid sequence of CDRL2 of anti-CD80 antibody (IgG2)
SEQ ID NO: 30: amino acid sequence of CDRL3 of anti-CD80 antibody (IgG2)
SEQ ID NO: 31: amino acid sequence of CDRH1 of anti-CD86 antibody (IgG2)
SEQ ID NO: 32: amino acid sequence of CDRH2 of anti-CD86 antibody (IgG2)
SEQ ID NO: 33: amino acid sequence of CDRH3 of anti-CD86 antibody (IgG2)
SEQ ID NO: 34: amino acid sequence of CDRL1 of anti-CD86 antibody (IgG2)
SEQ ID NO: 35: amino acid sequence of CDRL2 of anti-CD86 antibody (IgG2)
SEQ ID NO: 36: amino acid sequence of CDRL3 of anti-CD86 antibody (IgG2)
SEQ ID NO: 37: heavy chain nucleic acid sequence of chimeric anti-CD80 antibody
SEQ ID NO: 38: heavy chain amino acid sequence of chimeric anti-CD80 antibody
SEQ ID NO: 39: light chain nucleic acid sequence of chimeric anti-CD80 antibody
SEQ ID NO: 40: light chain amino acid sequence of chimeric anti-CD80 antibody
SEQ ID NO: 41: heavy chain nucleic acid sequence of chimeric anti-CD86 antibody
SEQ ID NO: 42: heavy chain amino acid sequence of chimeric anti-CD86 antibody
SEQ ID NO: 43: light chain nucleic acid sequence of chimeric anti-CD86 antibody
SEQ ID NO: 44: light chain amino acid sequence of chimeric anti-CD86 antibody
SEQ ID NO: 45: heavy chain variable region nucleic acid sequence of chimeric anti-CD80 antibody
SEQ ID NO: 46: heavy chain variable region amino acid sequence of chimeric anti-CD80 antibody
SEQ ID NO: 47: light chain variable region nucleic acid sequence of chimeric anti-CD80 antibody
SEQ ID NO: 48: light chain variable region amino acid sequence of chimeric anti-CD80 antibody
SEQ ID NO: 49: heavy chain variable region nucleic acid sequence of chimeric anti-CD86 antibody
SEQ ID NO: 50: heavy chain variable region amino acid sequence of chimeric anti-CD86 antibody
SEQ ID NO: 51: light chain variable region nucleic acid sequence of chimeric anti-CD86 antibody
SEQ ID NO: 52: light chain variable region amino acid sequence of chimeric anti-CD86 antibody
SEQ ID NO: 53: amino acid sequence of CDRH1 of chimeric anti-CD80 antibody
SEQ ID NO: 54: amino acid sequence of CDRH2 of chimeric anti-CD80 antibody
SEQ ID NO: 55: amino acid sequence of CDRH3 of chimeric anti-CD80 antibody
SEQ ID NO: 56: amino acid sequence of CDRL1 of chimeric anti-CD80 antibody
SEQ ID NO: 57: amino acid sequence of CDRL2 of chimeric anti-CD80 antibody
SEQ ID NO: 58: amino acid sequence of CDRL3 of chimeric anti-CD80 antibody
SEQ ID NO: 59: amino acid sequence of CDRH1 of chimeric anti-CD86 antibody
SEQ ID NO: 60: amino acid sequence of CDRH2 of chimeric anti-CD86 antibody
SEQ ID NO: 61: amino acid sequence of CDRH3 of chimeric anti-CD86 antibody
SEQ ID NO: 62: amino acid sequence of CDRL1 of chimeric anti-CD86 antibody
SEQ ID NO: 63: amino acid sequence of CDRL2 of chimeric anti-CD86 antibody
SEQ ID NO: 64: amino acid sequence of CDRL3 of chimeric anti-CD86 antibody

1. An antibody or variant thereof that inhibits an interaction between CD80 and/or CD86 expressed on a surface of a cell, and CD28 expressed on a surface of another cell, wherein the antibody or variant thereof does not substantially induce the production of a cytokine by immunostimulation.

2. The antibody or variant thereof of claim 1, wherein the cytokine comprises interferon γ.

3. The antibody or variant thereof of claim 1, which is a chimeric antibody.

4. The antibody or variant thereof of claim 1, wherein a subclass of the antibody is IgG1.

5. The antibody or variant thereof of claim 1, having a heavy chain with the amino acid sequence set forth in SEQ ID NO: 38 or 42,and a light chain with the amino acid sequence set forth in SEQ ID NO: 40 or 44.

6. The antibody or variant thereof of claim 1, comprising:
(a) a VH comprising CDRH1 of the amino acid sequence set forth in SEQ ID NO: 53, CDRH2 of the amino acid sequence set forth in SEQ ID NO: 54, and CDRH3 of the amino acid sequence set forth in SEQ ID NO: 55, and a VL comprising CDRL1 of the amino acid sequence set forth in SEQ ID NO: 56, CDRL2 of the amino acid sequence set forth in SEQ ID NO: 57, and CDRL3 set forth in SEQ ID NO: 58; or
(b) a VH comprising CDRH1 of the amino acid sequence set forth in SEQ ID NO: 59, CDRH2 of the amino acid sequence set forth in SEQ ID NO: 60, and CDRH3 of the amino acid sequence set forth in SEQ ID NO: 61, and a VL comprising CDRL1 of the amino acid sequence set forth in SEQ ID NO: 62, CDRL2 of the amino acid sequence set forth in SEQ ID NO: 63, and CDRL3 set forth in SEQ ID NO: 64.

7. The antibody or variant thereof of claim 6, comprising:
(a) a VH having the amino acid sequence set forth in SEQ ID NO: 46 or a variant sequence thereof, and a VL having the amino acid sequence set forth in SEQ ID NO: 48 or a variant sequence thereof; or
(b) a VH having the amino acid sequence set forth in SEQ ID NO: 50 or a variant sequence thereof, and a VL having the amino acid sequence set forth in SEQ ID NO: 52 or a variant sequence thereof.

8. The antibody or variant thereof of claim 1, wherein an Fc moiety of the antibody is a moiety that does not substantially induce the production of a cytokine by immunostimulation.

9. The antibody or variant thereof of claim 1, wherein a subclass of the antibody is IgG2 or IgG4.

10. The antibody or variant thereof of claim 9, wherein the subclass of the antibody is IgG4.

11. The antibody or variant thereof of claim 1, wherein the variant of the antibody is a variant lacking an Fc moiety of the antibody.

12. The antibody or variant thereof of claim 11, wherein the variant of the antibody is an Fab antibody, an F(ab′)₂ antibody, an Fab′ antibody, an Fv antibody, or an scFv antibody.

13. The antibody or variant thereof of claim 1, having an ability to induce immune tolerance.

14. The antibody or variant thereof of claim 1, wherein the cell expressing CD80 and/or CD86 is an antigen presenting cell, and the another cell expressing CD28 is a T cell.

15. The antibody of claim 1, wherein the antibody or variant thereof is an antagonistic anti-CD80 antibody, an antagonistic anti-CD86 antibody, an antagonistic anti-CD28 antibody, or an antagonistic bispecific antibody to CD80 and CD86, or a variant thereof.

16. The antibody or variant thereof of claim 1, which is a humanized antibody or a human antibody, or a variant thereof.

17. The antibody or variant thereof of claim 1, comprising an Fc moiety of IgG2 or IgG4 of a human antibody.

18. The antibody or variant thereof of claim 1, comprising an Fc moiety of IgG4 of a human antibody.

19. The antibody or variant thereof of claim 1, comprising:
(a) a VH comprising CDRH1 of the amino acid sequence set forth in SEQ ID NO: 25, CDRH2 of the amino acid sequence set forth in SEQ ID NO: 26, and CDRH3 of the amino acid sequence set forth in SEQ ID NO: 27, and a VL comprising CDRL1 of the amino acid sequence set forth in SEQ ID NO: 28, CDRL2 of the amino acid sequence set forth in SEQ ID NO: 29, and CDRL3 set forth in SEQ ID NO: 30; or
(b) a VH comprising CDRH1 of the amino acid sequence set forth in SEQ ID NO: 31, CDRH2 of the amino acid sequence set forth in SEQ ID NO: 32, and CDRH3 of the amino acid sequence set forth in SEQ ID NO: 33, and a VL comprising CDRL1 of the amino acid sequence set forth in SEQ ID NO: 34, CDRL2 of the amino acid sequence set forth in SEQ ID NO: 35, and CDRL3 set forth in SEQ ID NO: 36.

20. The antibody or variant thereof of claim 19, comprising:
(a) a VH having the amino acid sequence set forth in SEQ ID NO: 18 or a variant sequence thereof, and a VL having the amino acid sequence set forth in SEQ ID NO: 20 or a variant sequence thereof; or
(b) a VH having the amino acid sequence set forth in SEQ ID NO: 22 or a variant sequence thereof, and a VL having the amino acid sequence set forth in SEQ ID NO: 24 or a variant sequence thereof.

21-41. (canceled)
